(19) United States
(12) Patent Application Publication
(10) Pub. No.: US 2025/0099444 A1
(43) Pub. Date: Mar. 27, 2025

(54) Methods of Treating a Subject for Long-Haul COVID-19, and Compositions for Use in the Same

(71) Applicant: IncellDx, Inc.

(72) Inventors: Bruce K. Patterson, Menlo Park, CA (US); Edgar B. Francisco, San Carlos, CA (US); Amruta Pise, San Carlos, CA (US); Emily Long, San Carlos, CA (US)

(21) Appl. No.: 18/833,255

(22) PCT Filed: Jan. 23, 2023
(86) PCT No.: PCT/US2023/011309

§ 371 Date: Jul. 25, 2024

Related U.S. Application Data
(60) Provisional application No. 63/304,238, filed on Jan. 28, 2022.
(60) Provisional application No. 63/339,321, filed on May 6, 2022.

(51) Int. Cl. A61K 31/439; A61K 31/22; A61K 45/06; A61P 43/00

(52) U.S. Cl. CPC A61K 31/439; A61K 31/22; A61K 45/06; A61P 43/00

(57) ABSTRACT

Methods of treating a subject for long-haul COVID are provided. Aspects of the methods include administering to the subject a tropane CCR5/CCL5 interaction inhibitor in combination with a statin to treat the subject for long-haul COVID. Also provided are compositions for use in practicing the methods.

### CROSS-REFERENCE TO RELATED APPLICATIONS

Pursuant to 35 U.S.C. § 119 (e), this application claims priority to the filing dates of U.S. Provisional Patent Application Ser. No. 63/304,238 filed Jan. 28, 2022, and U.S. Provisional Patent Application Ser. No. 63/339,321 filed May 6, 2022, the disclosures of which applications are herein incorporated by reference.

### INTRODUCTION

Severe acute respiratory syndrome coronavirus 2 (SARS-CoV-2), the causative agent of coronavirus disease 2019 was first reported in December 2019. Since the initial cases of COVID-19 were reported from Wuhan, China in December 2019 (Huang, C. et al. Clinical features of patients infected with 2019 novel coronavirus in Wuhan, China. Lancet 395, 497-506 (2020)), SARS-CoV-2 has emerged as a global pandemic with an ever-increasing number of severe cases requiring invasive external ventilation that threatens to overwhelm health care systems (World Health Organization. Coronavirus disease (COVID-2019) situation reports. See website made up of “https://www.” before “who.int/emergencies/disease/novel-coronavirus-2019/situation-reports”).

The COVID-19 pandemic has had an immense detrimental impact on human health worldwide. COVID-19, the disease resulting from an infection with the coronavirus SARS-CoV-2, demonstrates a cause for great concern regarding the health of the global population, tallying over 300 million infections and upwards of 5.5 million deaths on record worldwide, with the growth of these numbers being imminent.¹ Furthermore, the progression of the pandemic has been facilitated by the spread of extremely infectious viral variants, including the Delta and Omicron variants, demonstrating the potential longevity of the COVID-19 pandemic, especially if the virus eventually becomes endemic. ¹ https://www.who.int/health-topics/coronavirus#tab=tab_1

Long-haul COVID, similarly referred to as long COVID, post-COVID conditions, post-acute COVID-19, and chronic COVID, is a condition that emerges following an infection with SARS-CoV-2. Long-haul COVID may impact an individual weeks post-infection and persist for months following.² Symptoms encompass a variety of afflictions ranging in extremity, such as fatigue, shortness of breath, cough, “brain fog,” chest pain, stomach pain, headache, and sometimes in more concerning instances, multiorgan effects and autoimmune conditions.² Unfortunately, as many as 30% of those with COVID-19 will exhibit symptoms of long-haul COVID.³ ² https://www.cdc.gov/coronavirus/2019-ncov/long-term-effects/index.html³ https://www.frontiersin.org/articles/10.3389/fimmu.2021.700782/full#T1b

Maraviroc, also known by brand name Selzentry, is a drug notably used in HIV treatment which functions as a CCR5 antagonist by inhibiting CCR5 receptors on cells. The use of Maraviroc in individuals with R5-tropic HIV-1, a strain of HIV which attacks cells via the CCR5 receptor, is accepted by the U.S. Food and Drug Administration, the European Commission, and Health Canada, among others.⁴ Notable clinical trials involving Maraviroc include MOTIVATE 1 and MOTIVATE 2, both of which exhibited Maraviroc's ability to limit the effects of the HIV virus, eliciting lower levels of HIV viral loads and even succeeding in some individuals with R5-tropic HIV-1 who had less favorable starting conditions, such as larger viral loads and reduced CD4 count.⁵ Furthermore, a >5 year follow-up of maraviroc use in HIV patients from the MOTIVATE trials presented limited instances of death and other health threats such as hepatic failure and myocardial infarctions, reflecting positively on the safety of maraviroc used in a relatively longer-term.⁶ ⁴https://www.ncbi.nlm.nih.gov/pmc/articles/PMC4598208/⁵https://www.ncbi.nlm.nih.gov/pmc/articles/PMC4598208/⁶ https://www.ncbi.nlm.nih.gov/pmc/articles/PMC3893710/

Statins are drugs employed to reduce cholesterol levels. Statins include atorvastatin, known by the brand name Lipitor, simvastatin, known by brand name Zocor, and pravastain, known by brand name Pravachol, among others. In addition, statins are believed to minimize the risk of blood clots, heart disease, and stroke in users. Furthermore, the National Center for Health Statistics presented that in 2011-2012, out of the approximate 27.9% U.S. adults who were 40 or older and claimed use of medication for lowering cholesterol levels, a statin was included in the treatment for 93% of these individuals.⁷ A Johns Hopkins study in 2014 further confirmed the safety of statins employed in a longer-term context, asserting that in examining 20 years of data, statins overall exhibited positive health effects and minimal associated concerns.⁸ ⁷https://www.cdc.gov/nchs/products/databriefs/db177.htm#:˜:text=The%20use%20of%20statins%20increased,adults%20aged%2075%20and%20over.⁸ https://www.hopkinsmedicine.org/health/wellness-and-prevention/how-statin-drugs-protect-the-heart

Considering the prominence of long-haul COVID and its concerning symptoms among those infected with SARS-CoV-2, a suitable treatment for the many patients suffering from long-haul COVID is needed. Given the relatively recent nature of health concerns stemming from the COVID-19 pandemic, there are few prospective medical treatments available for these afflictions. Furthermore, with many health efforts directed at vaccine development or immediate treatments for infected individuals, little attention has been provided towards addressing the long-term effects of COVID-19. As such, as more individuals are infected with SARS-CoV-2 and fall victim to its long-term impacts, the development of a treatment for long-haul COVID is of critical importance.

### SUMMARY

Methods of treating a subject for long-haul COVID are provided. Aspects of the methods include administering to the subject a tropane CCR5/CCL5 interaction inhibitor in combination with a statin to treat the subject for long-haul COVID. Also provided are compositions for use in practicing the methods.

### BRIEF DESCRIPTION OF THE FIGURES

FIG. 1 is a correlation matrix using the Pearson's correlation coefficient which was prepared to identify the positive correlations between the different biomarkers (cytokines) in the dataset and the subjective scores present.

FIG. 2 provides a modified Rankin Scale for Neurological Disability.

FIG. 3 provides a composite Autonomic Symptom Scale 31 (COMPASS 31).

FIG. 4 provides a New York Heart Association Classification.

FIG. 5 provides a Medical Research Council (MRC) Dyspnea Scale.

FIG. 6 provides a Fatigue Severity Score (FSS).

FIG. 7 provides symptoms in POVIP separated by vaccine type.

FIG. 8 provides a schematic of the fine-tuned decision tree model implemented with CART (Supplementary FIG. 2). The plotted decision tree with the highest performance for class separation following fine-tuning with grid search and cross validation. Cytokine origination hubs are round, defining cytokine hubs are in rectangles and disease states are in hexagons. The resulting tree had a maximum depth of 6 splitting hubs, and used the Gini impurity criterion, which measures the probability value of misclassifying a randomly selected event or individual from the dataset if such an element were randomly labeled based on its class distribution in the dataset.

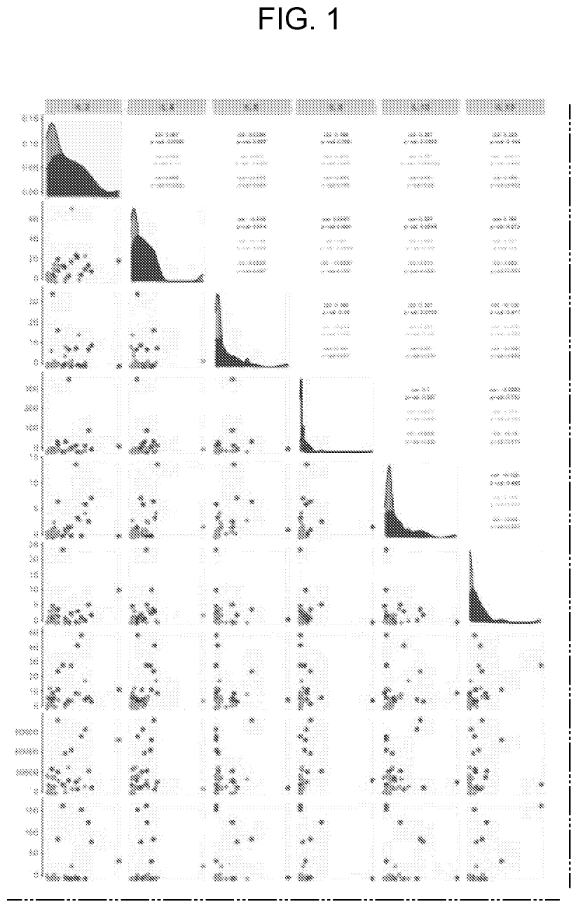

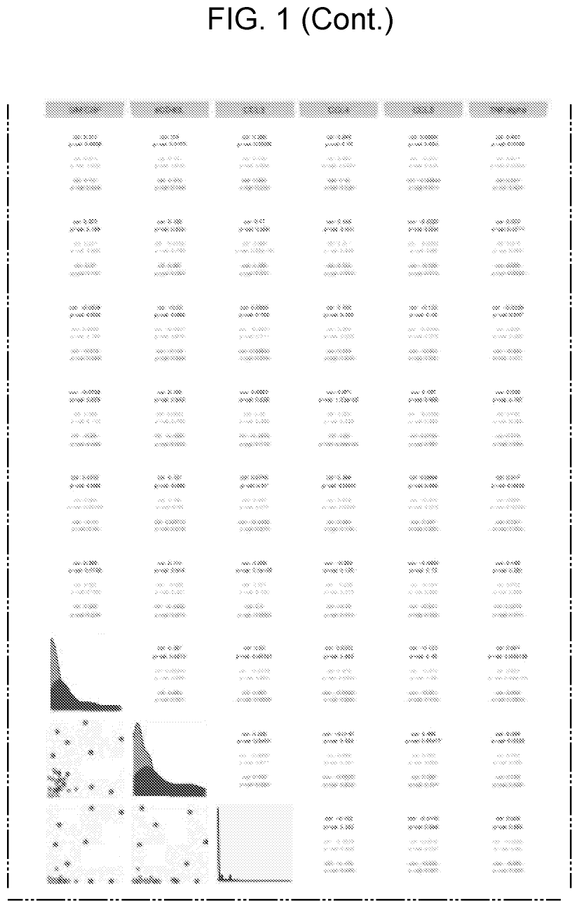

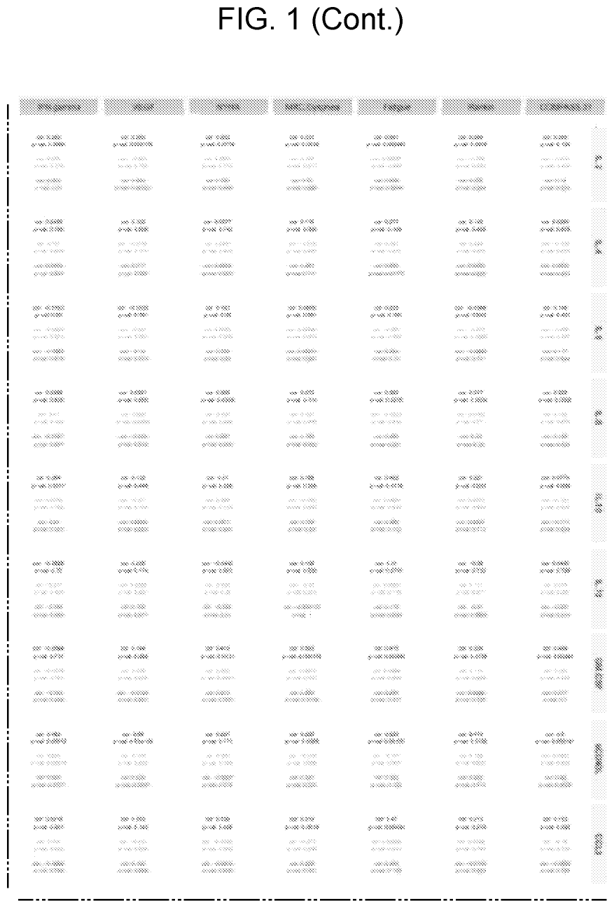

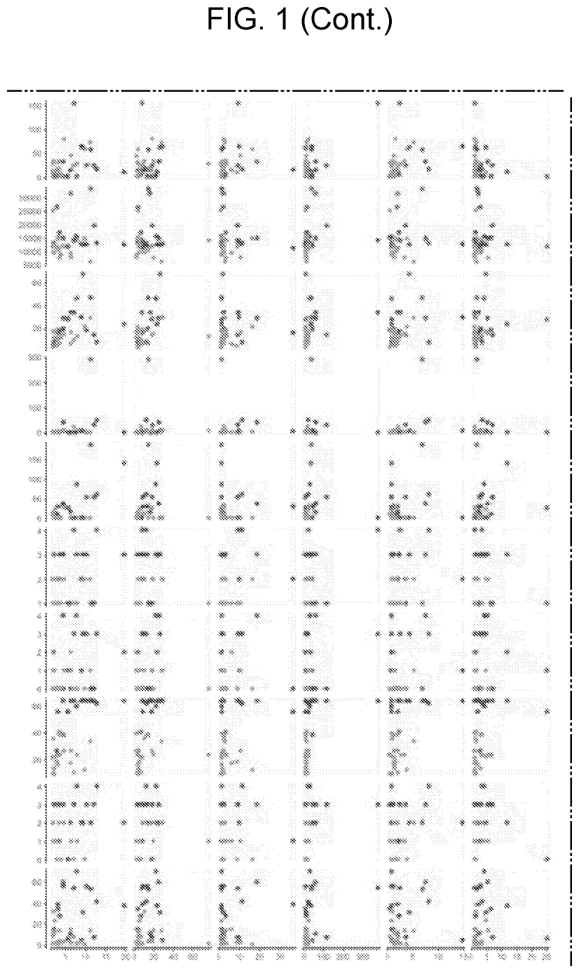

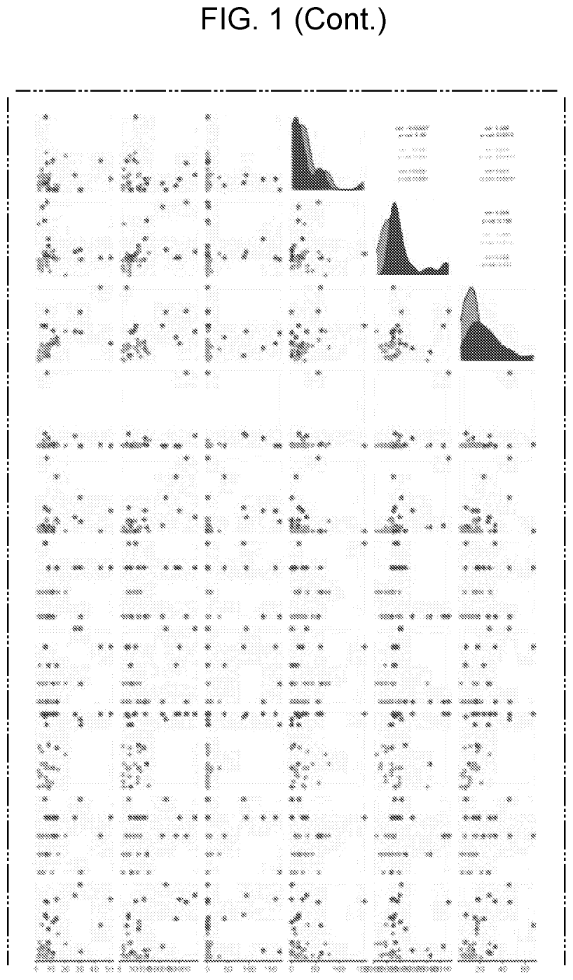

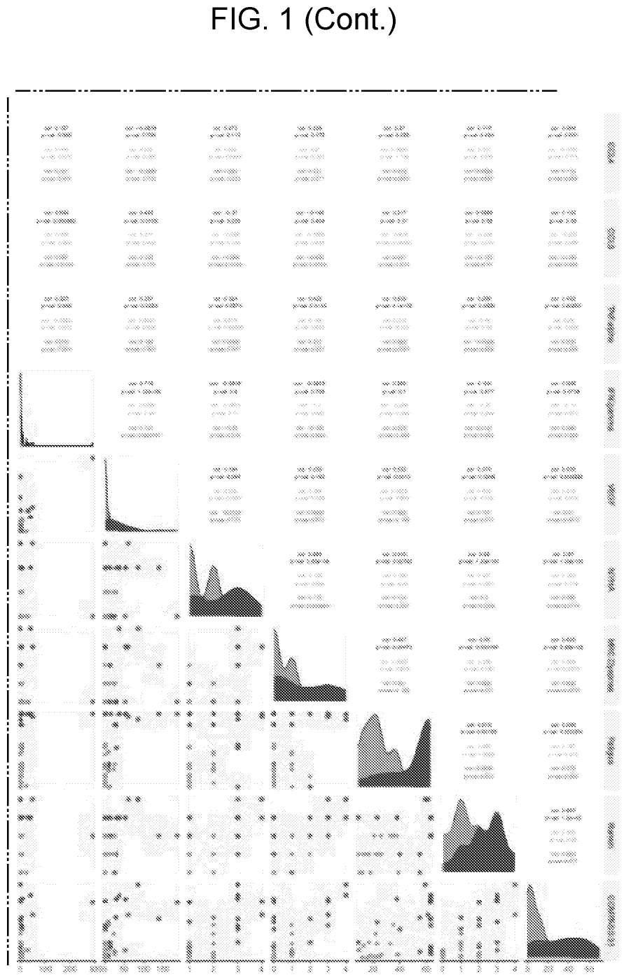

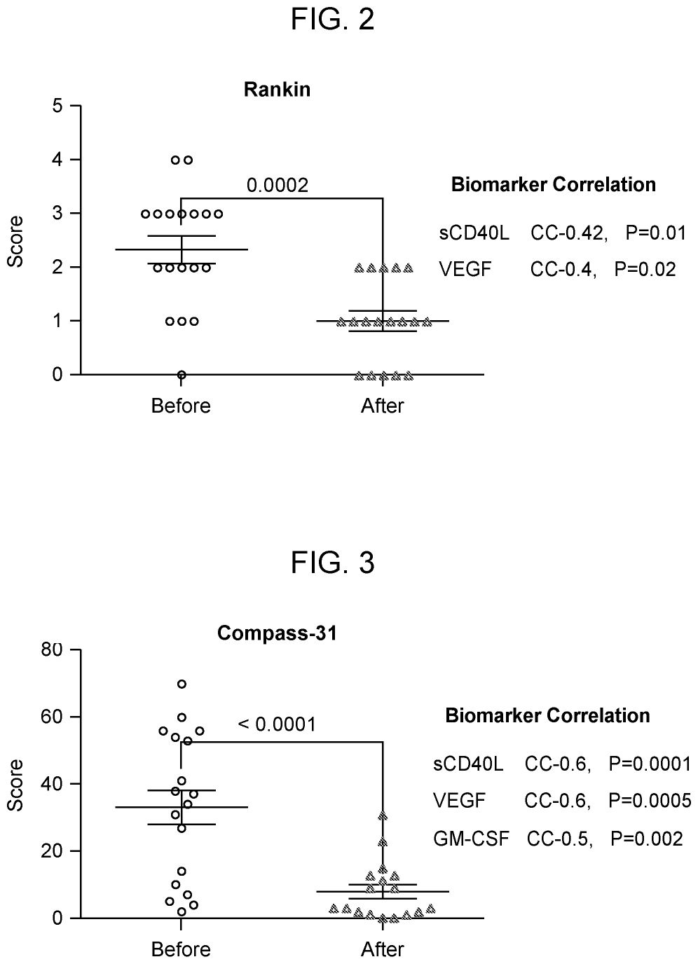

Supplementary FIGS. 1 and 2 provide additional information.

### DETAILED DESCRIPTION

Methods of treating a subject for long-haul COVID are provided. Aspects of the methods include administering to the subject a tropane CCR5/CCL5 interaction inhibitor in combination with a statin to treat the subject for long-haul COVID. Also provided are compositions for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, methods according to embodiments of the invention are discussed first in greater detail, followed by a review of compositions that find use in various embodiments of the methods.

### Methods

As summarized above, methods of treating a subject for long-haul COVID are provided. Long-haul COVID is also known as post-COVID conditions, long COVID, post-acute COVID-19, long-term effects of COVID, or chronic COVID. Symptoms encompass a variety of afflictions ranging in extremity, such as fatigue, shortness of breath, cough, “brain fog,” chest pain, stomach pain, headache, and sometimes in more concerning instances, multiorgan effects and autoimmune conditions. The term “treatment” or “treating” as used herein refers to the ability to ameliorate, suppress, mitigate, or eliminate one or more clinical symptoms of long-haul COVID. The effect may be prophylactic in terms of completely or partially preventing long-haul COVID outcomes and/or may be therapeutic in terms of partially or completely treating long-haul COVID symptoms.

Aspects of the methods include administering to the subject a tropane CCR5/CCL5 interaction inhibitor in combination with a statin to treat the subject for long-haul COVID. By “in combination with”, is meant that an amount of the tropane CCR5/CCL5 interaction inhibitor is administered anywhere from simultaneously to up to 5 hours or more, e.g., 10 hours, 15 hours, 20 hours or more, prior to, or after, the statin. In certain embodiments, the tropane CCR5/CCL5 interaction inhibitor and statin are administered sequentially, e.g., where the tropane CCR5/CCL5 interaction inhibitor is administered before or after the statin. In yet other embodiments, the tropane CCR5/CCL5 interaction inhibitor and statin are administered simultaneously, e.g., where the tropane CCR5/CCL5 interaction inhibitor and statin are administered at the same time as two separate formulations, or are combined into a single composition, that is administered to the subject. Regardless of whether the tropane CCR5/CCL5 interaction inhibitor and statin are administered sequentially or simultaneously, as illustrated above, or any effective variation thereof, the agents are considered to be administered together or in combination for purposes of the present invention. Routes of administration of the two agents may vary, where representative routes of administration are described in greater detail below.

The tropane CCR5/CCL5 interaction inhibitor employed in methods of the invention may vary. In some instances, the tropane CCR5/CCL5 interaction inhibitor is a compound of formula (I),

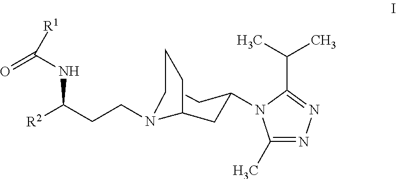

wherein R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms;
or a pharmaceutically acceptable salt or solvate thereof. In some instances, the tropane CCR5/CCL5 interaction inhibitor is a compound of formula (IA),

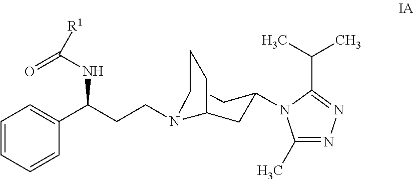

wherein R¹ represents either C₃₋₆ cycloalkyl optionally substituted by one or more fluorine atoms, or C₁₋₆ alkyl optionally substituted by one or more fluorine atoms, or a pharmaceutically acceptable salt or solvate thereof.
“C₁₋₆ alkyl” in the definition of R¹ includes straight-chain and branched groups. Examples of alkyl include methyl, ethyl, n-propyl, i-propyl, n-butyl, i-butyl, sec-butyl and t-butyl. “C₃₋₆ cycloalkyl” means cyclopropyl, cyclobutyl, cyclopentyl and cyclohexyl.

The compounds of formula (I) contain a basic center and suitable acid addition salts are formed from acids which form non-toxic salts. Examples include the hydrochloride, hydrobromide, hydroiodide, sulphate, bisulphate, nitrate, phosphate, hydrogen phosphate, acetate, maleate, fumarate, lactate, tartrate, citrate, gluconate, camsylate, succinate, saccharate, benzoate, methanesulphonate, ethanesulphonate, benzenesulphonate, p-toluenesulphonate and pamoate salts. Also of interest in certain embodiments are pharmaceutically acceptable solvates of the compounds of the formula (I) or salts thereof including the hydrates thereof. Also of interest in certain embodiments are polymorphs of the above compounds.

A compound of the formula (I) contains one or more asymmetric carbon atoms and therefore exists in two or more stereoisomeric forms. Of interest in certain embodiments are the individual stereoisomers of the compounds of the formula (I) and, where appropriate, the individual tautomeric forms thereof, together with mixtures thereof. Separation of diastereoisomers may be achieved by conventional techniques, e.g., by fractional crystallization, chromatography or H.P.L.C. of a stereoisomeric mixture of a compound of the formula (I) or a suitable salt or derivative thereof. An individual enantiomer of a compound of the formula (I) may also be prepared from a corresponding optically pure intermediate or by resolution, such as by H.P.L.C. of the corresponding racemate using a suitable chiral support or by fractional crystallization of the diastereoisomeric salts formed by; reaction of the corresponding racemate with a suitable optically active acid or base, as appropriate.

In some instances, R¹ is either C₄₋₆ cycloalkyl optionally substituted by one or two fluorine atoms, or C₁₋₄ alkyl optionally substituted by from one to three fluorine atoms.

In some instances, R¹ is either cyclobutyl, cyclopentyl, 4,4-difluorocyclohexyl or 3,3,3-trifluoropropyl. In some instances, R² is phenyl optionally substituted by 1 or 2 fluorine atom(s). In some instances, R² is phenyl or monofluorophenyl. In some instances, R² is phenyl or 3-fluorophenyl.

In some instances, tropane CCR5/CCL5 interaction inhibitor is: N-{(1S)-3-[3-(3-Isopropyl-5-methyl-4H-1,2,4-triazol-4-yl)-exo-8-azabicyclo[3.2.1]oct-8-yl]-1-phenylpropyl}cyclobutanecarboxamide; N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide; N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide; N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide; N-(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide: or a pharmaceutically acceptable salt or solvate of any thereof; or 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (Maraviroc); or a pharmaceutically acceptable salt or solvate of any thereof.

Additional details regarding tropane CCR5/CCL5 interaction inhibitors that may be employed in embodiments of the invention may be found in U.S. Pat. No. 6,667,314 the disclosure of which is herein incorporated by reference.

Any convenient statin may be employed in embodiments of the invention. Statins are compounds that inhibit HMG-COA reductase from catalyzing the conversion of HMG-COA to mevalonate, a rate-limiting step in the cholesterol biosynthetic pathway. Examples of statins that may be used in conjunction with the compositions and methods disclosed herein include, but are not limited to, atorvastatin or atorvastatin calcium (marketed as Lipitor® or Torvast®; see, e.g., U.S. Pat. No. 4,681,893 or 5,273,995) and atorvastatin combinations (e.g., atorvastatin plus amlodipine (marketed as Norvasc®), combination marketed as Caduet®, see, e.g., U.S. Pat. No. 6,455,574; atorvastatin plus CP-529414 (marketed as Torcetrapib®); atorvastatin plus APA-01; atorvastatin plus ezetimibe), cerivastatin (marketed as Lipobay® or Baycol®), fluvastatin (marketed as Lescol®; U.S. Pat. No. 4,739,073), lovastatin (marketed as Mevacor® or Altocor®; see, e.g., U.S. Pat. No. 4,231,938), lovastatin combinations (e.g., lovastatin plus Niaspan®, combination marketed as Advicor®), mevastatin, pitavastatin (marketed as Livalo® or Pitava®), pravastatin (marketed as Pravachol®, Mevalotin®, Selektine®, or Lipostat®; see, e.g., U.S. Pat. No. 4,346,227), pravastatin combinations (e.g., pravastatin plus fenofibrate), rosuvastatin (marketed as Crestor®), rosuvastatin combinations (e.g., rosuvastatin plus TriCor®), simvastatin (marketed as Zocor® or Lipex®; see, e.g., U.S. Pat. Nos. 4,444,784; 4,916,239; and 4,820,850), and simvastatin combinations (e.g., simvastatin plus ezetimibe, combination marketed as Vytorin®, see, e.g., U.S. Pat. No. 7,229,982; simvastatin plus Niaspan®, combination marketed as Simcor®; simvastatin plus MK-0524A, combination referred to as MK-0524B), as well as various pharmaceutically acceptable salts, solvates, salts, stereoisomers, prodrugs derivatives, or nitroderivatives of the compounds listed above. In some cases, such as for example with simvastatin, the active form of the statin is a metabolite formed in the body of a subject following administration. In other cases, statins are administered in their active form.

Where desired, active agents may be administered to a subject as a pharmaceutical composition. The active agent(s) may be administered to the subject using any convenient administration protocol capable of resulting in the desired activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means. With respect to the tropane CCR5/CCL5 interaction inhibitor, any convenient dosage may be employed. For oral or parenteral administration to human patients the daily dosage levels of compounds of formula (I), and their pharmaceutically acceptable salts, may be from 0.01 to 30 mg/kg (in single or divided doses) and in some instances from 0.01 to 15 mg/kg. Thus, tablets may contain 1 mg to 0.5 g of compound for administration singly or two or more at a time, as appropriate. With respect to the statins, any convenient dosage may be employed. In some embodiments, the statin therapy is a low, medium (i.e., moderate), or high intensity statin therapy. In some embodiments, the low intensity statin therapy includes about 5 mg to about 10 mg of simvastatin. In some embodiments, the medium intensity statin therapy includes about 5 mg to about 10 mg of rosuvastatin, about 10 mg to about 20 mg of atorvastatin, about 20 mg to about 40 mg of simvastatin, or about 10 mg to about 20 mg of simvastatin plus about 5 mg to about 10 mg of ezetimibe. In some embodiments, the high intensity statin therapy includes about 20 mg to about 40 mg rosuvastatin, about 40 mg to about 80 mg of atorvastatin, about 80 mg of simvastatin, or about 40 mg to about 80 mg of simvastatin plus about 5 mg to about 10 mg of ezetimibe. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

In practicing embodiments of the invention, active agent compositions may be administered according to any desired dosage, such as once per day, a few or several times per day, or even multiple times per day, depending upon, among other things, the indication being treated and the judgment of the prescribing physician. For example, in some instances, compositions that include one or more active agents may be administered once per day, a few or several times per day, or even multiple times per day, depending upon, among other things, the indication being treated and the judgment of the prescribing physician.

Depending on whether systemic and/or local treatment is chosen, methods of administration may be chosen depending also on the condition being treated and the pharmaceutical composition being administered. Administration of an effective amount (in one or multiple doses) of the subject agent(s) can be done in a variety of ways, including, but not limited to, subcutaneously, intravenously, intraperitoneally, intramuscularly, and direct injection to specified organs or tissues, systemic administration, etc. Administration of the pharmaceutical compositions may be through a single route or concurrently by several routes. As such, the active agent can be administered to a subject via a suitable route of administration and include oral, mucosal (e.g., nasal, sublingual, vaginal, buccal, or rectal), parenteral (e.g., subcutaneous, intravenous, bolus injection, intramuscular, or intra-arterial, intraperitoneal), or transdermal.

In those embodiments where an effective amount of an active agent is administered to the subject, the amount or dosage is effective when administered for a suitable period of time so as to evidence a reduction in one or more symptoms of the target disease. In some instances, an effective amount or dose of active agent will not only slow or halt the progression of the disease condition but will also induce the reversal of the condition, i.e., will cause an improvement in the subject's condition. Where desired, effectiveness of treatment may be assessed using any convenient protocol. Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, one or more target symptoms of the disease condition.

In some embodiments, the methods include assessing treatment efficacy of the combination therapy. Assessment of treatment efficacy may include evaluation of one or more symptoms of the subject. In some instances, the methods include assessing treatment efficacy by determining whether the subject maintains the long-haul COVID pathological type. In some instances, long-haul COVID pathological type is evaluated using the protocols described in U.S. patent application Ser. No. 17/351,055 published as US20210373034; the disclosure of which is herein incorporated by reference. For example, during treatment of a subject having a long-haul COVID pathological type, embodiments of the methods may include further assessing the subject to determine efficacy of the treatment. For example, a subject may be assessed one or more times following treatment to determine whether the subject should still be assigned as having the long-haul COVID pathological type, or whether subject no longer has the long-haul COVID pathological type. In such an embodiment, the subject may be assessed to determine whether the subject still has a long-haul COVID pathological score, e.g., S1. The determination of the pathological type or absence thereof may be employed as a measure or evaluation of the therapeutic treatment regimen being administered to the subject. In such embodiments, the frequency of assaying may vary, such as daily, every two days, weekly, every two weeks, etc.

In some instances, the methods include determining when to administer therapy to the subject if the subject is assigned a long-haul COVID pathological type. The methods described in U.S. patent application Ser. No. 17/351,055 published as US20210373034; the disclosure of which is herein incorporated by reference, can also be used to determine the appropriate time to discontinue therapy or change therapy in the instance that the severity or long scores have not changed after therapy.

In some instances a cytokine hub mediated method, such as those described below, is employed to assess the presence of long haul COVID and/or monitor progression of disease treatment.

As such, methods of treating a subject for long-haul COVID are provided. Subjects suffering from long-haul COVID may be referred to as long haulers, and may suffer from one or more symptoms indicative of long-haul COVID. “Long Haulers” are subjects who experience a multitude of symptoms from several weeks to months after recovering from their acute illness and presumably months after viral clearance. These symptoms include joint pain, muscle aches, fatigue, “brain fog” and others. These symptoms can commonly resemble rheumatic diseases such as rheumatoid arthritis, autoimmune disorders, and others such as fibromyalgia and chronic fatigue syndrome (Chen et al., “Inflammatory responses and inflammation-associated diseases in organs”, Oncotarget 9, 7204-7218 (2018)). Many of these common disorders are caused by inflammation, hyper- and/or auto-immunity and some such as chronic fatigue are associated with viral persistence after an acute infection with pathogens such as Epstein Barr and Cytomegalovirus (Rasa et al., “Chronic viral infections in myalgic encephalomyelitis/chronic fatigue syndrome (ME/CFS)”, J Transl Med 16, 268 (2018)). Recent studies including those from our laboratory have suggested that (CC) may be caused by persistent COVID itself (Mudd et al., “SARS-CoV-2 viral RNA shedding for more than 87 days in an individual with an impaired CD8+ T-cell response”, Front Immunol (in press)). In embodiments of these methods, the subject may have been identified as a long hauler using the methods of assigning a COVID pathological type for a subject suffering from COVID-19, e.g., as described in U.S. patent application Ser. No. 17/351,055 published as US20210373034; the disclosure of which is herein incorporated by reference.

The terms “subject,” “individual,” “host,” and “patient,” are used interchangeably herein and refer to any mammalian subject for whom diagnosis, treatment, or therapy is desired, particularly humans. As reviewed above, by “treatment” it is meant that at least an amelioration of one or more symptoms associated with long-haul COVID afflicting the subject is achieved, where amelioration is used in a broad sense to refer to at least a reduction in the magnitude of a parameter, e.g., a symptom, associated with the impairment being treated. As such, treatment also includes situations where long-haul COVID, or at least symptoms associated therewith, is completely inhibited, e.g., prevented from happening, or stopped, e.g., terminated, such that the adult mammal no longer suffers from the impairment, or at least the symptoms that characterize the impairment.

### Formulations

Also provided are pharmaceutical compositions containing the tropane CCR5/CCL5 interaction inhibitor and/or the statin employed in the subject methods. Accordingly, the tropane CCR5/CCL5 interaction inhibitor and/or statin in pharmaceutical compositions, e.g., in the form of a pharmaceutically acceptable salt, can be formulated for oral, topical or parenteral administration for use in the subject methods, as described above. In certain embodiments, e.g., where the compounds are administered as separate formulations (such as in those embodiments where they are administered sequentially), separate or distinct pharmaceutical compositions, each containing a different active agent, are provided. In yet other embodiments, a single formulation that includes both of the tropane CCR5/CCL5 interaction inhibitor and the statin (i.e., one composition that includes both active agents) is provided.

By way of illustration, the tropane CCR5/CCL5 interaction inhibitor and/or the statin can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder.

For example, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example, polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example, by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example, liquid paraffin or fractionated coconut oil can be prepared and filled into a soft gelatin capsule.

Tablet binders that can be included are acacia, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. Additionally, it may be desirable to add a coloring agent to make the dosage form more attractive in appearance or to help identify the product.

The compounds of the invention and their pharmaceutically acceptable salts that are active when given parenterally can be formulated for intramuscular, intrathecal, or intravenous administration.

A typical composition for intramuscular or intrathecal administration will be of a suspension or solution of active ingredient in an oil, for example, arachis oil or sesame oil. A typical composition for intravenous or intrathecal administration will be a sterile isotonic aqueous solution containing, for example, active ingredient and dextrose or sodium chloride, or a mixture of dextrose and sodium chloride. Other examples are lactated Ringer's injection, lactated Ringer's plus dextrose injection, Normosol-M and dextrose, Isolyte E, acylated Ringer's injection, and the like. Optionally, a co-solvent, for example, polyethylene glycol, a chelating agent, for example, ethylenediamine tetracetic acid, and an anti-oxidant, for example, sodium metabisulphite may be included in the formulation. Alternatively, the solution can be freeze dried and then reconstituted with a suitable solvent just prior to administration.

The compounds of the invention and their pharmaceutically acceptable salts which are active on rectal administration can be formulated as suppositories. A typical suppository formulation will generally consist of active ingredient with a binding and/or lubricating agent such as a gelatin or cocoa butter or other low melting vegetable or synthetic wax or fat.

The compounds of this invention and their pharmaceutically acceptable salts which are active on topical administration can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, herein incorporated by reference in its entirety. Such patches may be constructed for continuous, pulsatile, or on demand delivery of pharmaceutical agents.

In certain embodiments of interest, the tropane CCR5/CCL5 interaction inhibitor and the statin are administered as a single pharmaceutical formulation, that, in addition to including an effective amount of the tropane CCR5/CCL5 interaction inhibitor and the statin, includes other suitable compounds and carriers, and may also be used in combination with other active agents. The present invention, therefore, also includes pharmaceutical compositions comprising pharmaceutically acceptable excipients. The pharmaceutically acceptable excipients include, for example, any suitable vehicles, adjuvants, carriers or diluents, and are readily available to the public. The pharmaceutical compositions of the present invention may further contain other active agents that are well known in the art.

One skilled in the art will appreciate that a variety of suitable methods of administering a formulation of the present invention to a subject or host, e.g., patient, in need thereof, are available, and, although more than one route can be used to administer a particular formulation, a particular route can provide a more immediate and more effective reaction than another route. Pharmaceutically acceptable excipients are also well-known to those who are skilled in the art and are readily available. The choice of excipient will be determined in part by the particular compound, as well as by the particular method used to administer the composition. Accordingly, there are a wide variety of suitable formulations of the pharmaceutical composition of the present invention. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, or in addition to the active ingredient, and other such carriers that are known in the art to be appropriate.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of means.

The dose administered to an animal, particularly a human, in the context of the present invention should be sufficient to cause a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to anticancer or immunosuppressive agents that are known to cause the desired growth inhibitory or immunosuppressive response.

Optionally, the pharmaceutical composition may contain other pharmaceutically acceptable components, such a buffers, surfactants, antioxidants, viscosity modifying agents, preservatives and the like. Each of these components is well-known in the art. For example, see U.S. Pat. No. 5,985,310, the disclosure of which is herein incorporated by reference.

Other components suitable for use in the formulations of the present invention can be found in Remington's Pharmaceutical Sciences, Mace Publishing Company, Philadelphia, Pa., 17th ed. (1985). In an embodiment, the aqueous solution of cyclodextrin also contains dextrose, e.g., about 5% dextrose.

### Kits

Also provided are kits that find use in embodiments of the invention. The kits may include a tropane CCR5/CCL5 interaction inhibitor and a statin, where the tropane CCR5/CCL5 interaction inhibitor and statin may be present as separate pharmaceutical compositions or present in a single pharmaceutical composition. The kit components may be present in packaging, which packaging may be sterile, as desired.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### I. Treatment of Long-Haul COVID Patients

Three long-haul COVID patients were treated with maraviroc 300 mg twice a day in addition to 10 mg of pravastatin once a day for six weeks. The results are provided in Table 1, below. In Table 1, the four markers highlighted in yellow represent biomarkers for the underlying vascular inflammation, one of, if not the major cause of, long-haul COVID or PASC. All four markers in the 3 patients were reduced which was accompanied by improvement in symptoms.

TABLE 1

| Average | | | | | GM- | | CCL5 | CCL3 | | | | | | CCL4 |
|---|---|---|---|---|---|---|---|---|---|---|---|---|---|---|
| (pg/ml) | TNF-α | IL-4 | IL-13 | IL-2 | CSF | sCD40L | (RANTES) | (MIP-1α) | IL-6 | IL-10 | IFN-γ | VEGF | IL-8 | (MIP-1β) |
| Normals | 9.09 | 4.18 | 3.94 | 6.17 | 51.27 | 7192.39 | 10781.84 | 22.82 | 2.21 | 0.67 | 1.94 | 9.32 | 16.87 | 76.84 |
| PT 1 Before | 4.1 | 2.7 | 3 | 2.2 | 5.2 | 60058 | 55209 | 1.22 | 14.8 | 1.5 | 1.9 | 53.7 | 32568 | 129 |
| After | 2.8 | 2.4 | 0.54 | 2 | 1.67 | 14691 | 10687 | 19.3 | 0.9 | 0.9 | 3.9 | 2.18 | 24.9 | 21.8 |
| PT 2 Before | 18.2 | 2.6 | 6 | 2.9 | 11.7 | 29117 | 15076 | 1.22 | 0.67 | 0.3 | 0.3 | 9.8 | 2 | 0.8 |
| After | 11.4 | 3.4 | 2.5 | 2 | 7.6 | 5407 | 9647 | 1.22 | 1.1 | 1.3 | 1.6 | 0.85 | 5.6 | 3.9 |
| PT 3 Before | 7.6 | 14 | 0.6 | 4.8 | 2.3 | 34665 | 13242 | 109 | 2.5 | 2.5 | 7.2 | 0.8 | 8.8 | 4.2 |
| After | 4.9 | 7 | 0.6 | 2.3 | 2.1 | 14554 | 12548 | 1.2 | 0.7 | 3.8 | 4 | 0.8 | 5.5 | 22.3 |

As such, the above demonstrates that maraviroc in combination with a statin, such as pravastatin, is an effective treatment for long-haul COVID patients.

### II. Targeting the Monocytic-Endothelial-Platelet Axis with Maraviroc and Pravastatin as a Therapeutic Option to Treat Long COVID/Post-Acute Sequelae of COVID (PASC)

### A. Abstract

Post-acute sequelae of COVID (PASC), or long COVID, is a multisystem complication of SARS-CoV-2 infection that continues to debilitate millions worldwide thus highlighting the public health importance of identifying effective therapeutics to alleviate this illness. The pathophysiology behind PASC may be attributed to the recent discovery of persistent S1 protein subunit of SARS-CoV-2 in CD16+ monocytes up to 15 months after infection. CD16+ monocytes, which express both CCR5 and fractalkine receptors (CX3CR1), play a role in vascular homeostasis and endothelial immune surveillance. We believe targeting these receptors using the CCR5 antagonist, maraviroc, along with pravastatin, could disrupt the monocytic-endothelial-platelet axis that may be central to the etiology of PASC. Using five validated clinical scales (NYHA, MRC Dyspnea, COMPASS-31, modified Rankin, and Fatigue Severity Score) to measure 18 participants' response to treatment, we observed significant clinical improvement in six to twelve weeks on a combination of maraviroc 300 mg PO BID and pravastatin 10 mg PO daily. Subjective neurological (p=0.002), autonomic (p<0.0001), respiratory (p=0.0153), cardiac (p=0.002) and fatigue (p<0.0001) symptoms scores all decreased which correlated with statistically significant decreases in vascular markers sCD40L and VEGF. These findings indicate that by interrupting the monocytic-endothelial-platelet axis, maraviroc and pravastatin may restore the immune dysregulation observed in PASC and could be potential therapeutic options.

### B. Introduction

Post-acute sequelae of COVID (PASC), commonly referred to as long COVID or chronic COVID, is an emerging public health syndrome that continues to devastate and debilitate adult and pediatric survivors of acute SARS-CoV-2 infection. The World Health Organization (WHO)-led Delphi consensus defined PASC as a syndrome starting three months from onset of probable infection with symptoms lasting over two months and could not be explained by an alternative diagnosis (1). Over 200 symptoms have been attributed to PASC (2) thus posing an enormous challenge clinically. The multi-organ involvement causes cognitive impairment, debilitating neuropathy, chronic migraines, autonomic dysfunction, cardiac dysrhythmias, dyspnea at rest, severe fatigue, and myalgias (3). Presently, minimal therapeutic options are available to treat PASC which can be attributed to the pathology not yet being fully described. However, we recently reported that the S1 protein subunit of SARS-CoV2 is retained in both nonclassical (CD14− CD16+) and intermediate (CD14+CD16+) monocytes several months after acute infection. Typically, these monocytes persist only for a few days, but in PASC patients, the S1 containing monocytes can persist for months and years (4), which we propose contributes to the pathophysiology behind PASC. Nonclassical monocytes are involved in phagocytosis and vascular adhesion by patrolling the endothelium under homeostatic and inflammatory conditions through B2 integrin, lymphocyte function-associated antigen-1 (LFA-1) and high levels of fractalkine receptors (CX3CR1) (5,6). On the other hand, CD14+CD16+ monocytes express high levels of C-C chemokine receptor type 5 (CCR5) and fractalkine receptors and are involved in antigen presentation, cytokine secretion and apoptosis regulation (6,7). Since CCR5 and fractalkine receptors have been studied for various chronic inflammatory pathologies, we hypothesized that these receptors may also be therapeutic targets for PASC. CD16+ monocytes also produce high levels of various pro-inflammatory cytokines which could be an explanation for the heterogenous symptomatology in PASC. Specifically, elevations in C-C chemokine ligand 5 (CCL5)/RANTES (Regulated on Normal T-cell Expression and Secretion), IL-2, IL-6, IFN-gamma and Vascular Endothelial Growth Factor (VEGF), along with decrease in CCL4 have been observed in patients and are hypothesized to be contributing to the pathophysiology of PASC (8).

Here, we describe an 18 participant case series investigating the combination of the CCR5 receptor antagonist maraviroc, and pravastatin, which targets fractalkine, as a potential therapeutic approach in addressing and treating the potential pathology of PASC. The CCR5 receptor is a seven-transmembrane G protein-coupled receptor (GPCR) that is found on macrophages and T-lymphocytes and functions to regulate trafficking and effector functions of these cells (9). The role of CCR5 as a co-receptor for human immunodeficiency virus (HIV) entry was discovered in 1996. Maraviroc is the first and only US Food and Drug Administration (FDA) and European Medical Agency (EMA) approved CCR5 receptor antagonist available to date. Maraviroc is a negative allosteric modulator of the CCR5 receptor, and by binding to the CCR5 receptor, it induces receptor conformational changes that prevent the chemokine binding of RANTES (CCL5) and CCR5-mediated signaling (10). While this mechanism has been researched and studied extensively in HIV infection, there is increasingly greater recognition and appreciation of the CCR5-CCL5 axis in many other conditions and pathologies such as cancer, autoimmune disorders and endothelial dysfunction. This signaling is central to the pathophysiology of inflammation by directing immune cells through a process called chemotaxis. These actions are mediated through RANTES, which is produced by platelets, macrophages, eosinophils, fibroblasts, endothelial, epithelial and endometrial cells. (11). The effects of RANTES have been implicated in respiratory tract infections, especially viruses possessing RNA genome (including coronavirus, influenza, RSV and adenovirus), asthma, neuroinflammation, and atherosclerosis (12,13). Maraviroc has also been documented to restore the homeostasis of regulatory T-cells (Treg), increase CD4 and CD8 positive counts, and inhibit HIV-associated chronic inflammation and activation (14,15). Interestingly, CD4 and CD8 positive T-cells expressing PD-1 and T-regs have been observed to be significantly lower in PASC patients compared to healthy controls (8), thus suggesting maraviroc could restore the immune dysregulation seen in PASC. The commonly known mechanism of action of statins is inhibition of hydroxymethylglutaryl-CoA (HMG-COA) reductase enzyme in lowering cholesterol. However, statins have also been implicated in reducing inflammation, suppressing fractalkine, and lowering VEGF and IL-6 (16), and as such, may play a role in the pathophysiology of PASC. We targeted fractalkine using pravastatin since CD16+ monocytes express high levels of the fractalkine receptor believing this may address the elevations in vascular markers seen in PASC.

### C. Methods/Material

After written informed consent was obtained, the medical records and immunological lab reports from 17 adult and one pediatric PASC participants from the Chronic COVID Treatment Center treated with maraviroc 300 mg PO BID daily and pravastatin 10 mg PO daily were collected and analyzed.

### 1. Inclusion Criteria

All the participants in the case series were COVID-19 survivors with documented FDA EUA approved RT-PCR SARS-CoV2 positive test and/or were positive for anti-SARS-CoV2 antibodies using FDA EUA approved tests. All participants had one or more new onset symptoms that persisted greater than three months after the diagnosis of acute COVID-19 infection. These symptoms included cognitive impairment (brain fog), migraines, post exertional malaise (PEM), myalgias, arthralgias, severe fatigue, tachyarrhythmias, postural orthostatic tachycardia syndrome (POTS) and shortness of breath. All participants displayed either isolated or combinations of elevated pro-inflammatory markers: RANTES, TNF-alpha, IFN-gamma, sCD40L, VEGF, IL-6, IL-2 and IL-8 on the IncellKINE panel. The IncellKINE cytokine panel is a set of 14 cytokines that was constructed from a machine-based learning algorithm that identified potential markers of PASC.

### 2. Exclusion Criteria

We excluded participants with a history of migraines, neuropathy, inflammatory bowel disease, depression and anxiety disorders, chronic fatigue syndrome, fibromyalgia, arthritis, COPD, asthma, chronic kidney disease, chronic heart failure (CHF), arrhythmias, bleeding disorders, and anticoagulation therapy prior to COVID-19 infection.

### 3. Validated Scoring System for Patient Assessment Before and After Treatment

A challenge in studying and defining PASC is the heterogenous clinical presentation and multisystem involvement. Thus, we categorized the main participant symptoms into 5 groups: neurological/autonomic function, cardiac, respiratory, overall functionality and fatigue. Since there are no validated scales for PASC, we used five validated scales for other organ systems (New York Heart Association (NYHA), Modified Rankin Scale for Neurologic Disability, Fatigue Severity Scale (FSS), COMPASS-31 and Medical Research Council (MRC) Dyspnea Scale, respectively) to measure subjective participant responses to treatment. Participants were administered validated self-questionnaires about their PASC symptoms before and after treatment with maraviroc and pravastatin treatment. The length of duration of treatment varied based on repeat immune markers and participant-reported symptom improvement. Since many of these participants were on other medications and anti-inflammatories prior to starting maraviroc and pravastatin, the biomarkers and subjective data presented are from the onset of this combination. Phone interviews were conducted with each participant before and after subjective responses to the medications.

The New York Heart Association (NYHA) Functional Classification was used to classify severity of PASC associated cardiac symptoms.

| | | | | | | | |
|---|---|---|---|---|---|---|---|
| Class | No limitation of | Class | Slight limitation of | Class | Marked limitation of | Class | Unable to carry on any |
| 1 | physical activity. | 2 | physical activity. | 3 | physical activity. | 4 | physical activity |
| | Ordinary physical | | Comfortable at rest. | | Comfortable at rest. | | without discomfort. |
| | activity does not | | Ordinary physical activity | | Less than ordinary | | Symptoms of heart |
| | cause undue | | results in fatigue, | | activity causes fatigue, | | failure at rest. If any |
| | fatigue, palpitation, | | palpitation, dyspnea | | palpitation, or dyspnea. | | physical activity is |
| | dyspnea (shortness | | (shortness of breath). | | | | undertaken, |
| | of breath). | | | | | | discomfort increases. |

The Composite Autonomic Symptom Scale 31 (COMPASS 31), a self-rating questionnaire consisting of 31 items and evaluating orthostatic intolerance, vasomotor, secretomotor, gastrointestinal, bladder, and pupillomotor function, was used to measure autonomic dysfunction and the subsequent therapeutic effects of maraviroc and pravastatin. A sub raw score for each of the six domains was calculated and converted into a weighted sub-score. The sum of this weighted sub-score gave a total score which ranged from 0 to 100, with 0 meaning no autonomic symptoms and 100 reflecting the most severe autonomic symptoms.

Medical Research Council (MRC) Dyspnea Scale is a validated method comprised of five statements that aims to measure perceived feeling of breathlessness.

| | | | | | | | | | |
|---|---|---|---|---|---|---|---|---|---|
| Grade | Are you ever | Grade | Are you short | Grade | Do you have to | Grade | Do you have to | Grade | Are you too |
| 1 | troubled by | 2 | of breath when | 3 | walk slower | 4 | stop for breath | 5 | breathless to |
| | breathlessness | | hurrying on | | than most | | after walking | | leave the house, |
| | except on | | the level or | | people on the | | about 100 yds. | | or breathless |
| | strenuous | | walking up | | level? Do you | | (or after a few | | after undressing? |
| | exertion? | | a slight hill? | | have to stop | | minutes) on the | | |
| | | | | | after a mile or | | level? | | |
| | | | | | so (or after 15 | | | | |
| | | | | | minutes) on | | | | |
| | | | | | the level at | | | | |
| | | | | | your own pace? | | | | |

The Modified Rankin Scale for Neurologic Disability is a validated scale to measure degree of disability after suffering a stroke or neurological insult.

| | | | | | | | | | | | |
|---|---|---|---|---|---|---|---|---|---|---|---|
| 0 | no | 1 | No significant | 2 | Slight disability; | 3 | Moderate disability; | 4 | Moderately severe | 5 | Severe disability; |
| | symptoms | | disability despite | | unable to carry | | requiring some help, | | disability; unable | | bedridden, |
| | | | symptoms; able to | | out all previous | | but able to walk | | to walk without | | incontinent and |
| | | | carry out all usual | | activities, but | | without assistance | | assistance and | | requiring constant |
| | | | duties and activities | | able to look after | | | | unable to attend to | | nursing care |
| | | | | | own affairs | | | | own bodily needs | | and attention |
| | | | | | without assistance | | | | without assistance | | |

The Fatigue Severity Scale (FSS) questionnaire is a nine-statement validated scale that rates the severity of fatigue symptoms. Participants were asked how accurately each statement reflected their condition before and after treatment with maraviroc and pravastatin and the extent to which they agreed or disagreed based on a scale of 1 (strongly disagree) to 7 (strongly agree).

| | | | | | | | | | | | |
|---|---|---|---|---|---|---|---|---|---|---|---|
| 1 | My motivation | 2 | Exercise brings | 3 | I am easily | 4 | Fatigue interferes | 5 | Fatigue causes | 6 | My fatigue prevents |
| | is lower when I | | on excessive | | fatigued. | | with my physical | | frequent problems | | sustained physical |
| | am fatigued. | | fatigue. | | | | functioning. | | for me. | | functioning |

| | | | | | |
|---|---|---|---|---|---|
| 7 | Fatigue interferes | 8 | Fatigue is among | 9 | Fatigue interferes |
| | with carrying out | | my three most | | with my work, |
| | certain duties and | | disabling symptoms. | | family, or social |
| | responsibilities. | | | | life. |

### 4. Serum Cytokine Measurements from Participants: Multiplex Cytokine Quantification

Fresh plasma was used for cytokine quantification using a customized 14-plex bead based flow cytometric assay (IncellKINE, IncellDx, Inc) on a CytoFlex flow cytometer as previously described (8) using the following analytes: ‘TNF-α’, ‘IL-4’, ‘IL-13’, IL-2′, ‘GM-CSF’, ‘sCD40L’, ‘CCL5 (RANTES)’, ‘CCL3 (MIP-1α)’,′IL-6′, ‘IL-10’, ‘IFN-γ’, ‘VEGF’, ‘IL-8’, and ‘CCL4 (MIP-1β). For each participant sample, 25 μL of plasma was used in each well of a 96-well plate.

### 5. Data Acquisition and Preprocessing

The dataset was acquired in a Microsoft Excel (xlsx) table format, consisting of a total of 22 columns representing different features. The features or columns were organized in the following order:
Anonymized participant ID (column 1)
Weeks on medication (column 2)
Status of participant-before or after treatment (column 3)
Cytokine biomarker profiles (columns 4-17)
Subjective scores (columns 18-22)

In total there were 18 unique individuals, with each individual being represented in duplicate for before and after treatment. The presence of a pre and post treatment for each individual categorized as PASC allowed us the possibility to separate the data set into a before and after data sets for the required statistical comparisons. To separate the before and after groups, we used the python programming language (version 3.9) and the pandas library (18,19), which allowed us to group the samples according to before and after treatment. Once we separated the data in the two data sets, we then conducted the necessary comparative statistical analysis, including the statistical test to determine if there were significant differences between the two groups.

### 6. Wilcoxon's Paired Test to Compare the Before and After Treatment Groups

To determine if there were differences between the biomarker's levels of the two groups (before and after) we decided to compare the datasets by implementing the non-parametric Wilcoxon's paired test. The implementation of this test was done using the python library scipy (20). The selection of the Wilcoxon test was based on the assumption that this non-parametric test does not assume normal distribution of the variables. Additionally, in contrast to parametric tests like ANOVA, Wilcoxon's paired test does not base its comparison on the mean but median values. For our data we compared group before and group after with two alternative hypotheses. The first was a two-sided test, which resulted in a p-value less than 0.05. Subsequently, we tested for an alternative hypothesis “greater”, resulting in a p-value of less than 0.05.

### 7 Correlation Analysis Between Biomarker Levels and Subjective Scores

In order to identify potential statistically significant relationships between the biomarkers present in the dataset and the subjective scores, we imported the full dataset into the R programming language (version 4.1.1) (21) and conducted a correlation analysis. The correlation analysis was calculated using the Pearson correlation coefficient, which allows the measurement of both strength and direction of the linear relationship between two variables.

The Pearson correlation coefficient has the advantage that its values are highly interpretable, always ranging from −1 (strong negative correlation) to +1 (strong positive correlation). Correlation coefficients were calculated for both the before and after data points, and to validate their statistical significance, their p-value was calculated. We defined that correlation coefficients were statistically significant if their p-value was equal or less than 0.05. In order to properly interpret and convey the correlation relationships and their statistical significances, we constructed a modified pair plot with the R package GGally and additional functions to plot the p-values for the correlation coefficients. GGally is an extension to the R package ggplot2 (versions 2.1.2 and 3.3.5 respectively) (22). The pair plot presented was color coded for each group (blue=before, red=after) and consisted of scatterplots of each variable in the dataset for both the before and after groups, a density plot (a smooth representation of a histogram to approximate the distribution of each group), and the correlations for each group as well as the joint correlation. For the correlation coefficients, the p-values were added under the Pearson's correlation coefficient and maintain the color code scheme, with the addition of black representing the joint correlation.

### 8. Validation of Long Hauler Status Using a Machine Learning Classifier

The individuals in the dataset were identified as a long hauler (someone diagnosed with PASC). In order to further validate this classification, we implemented our previously reported machine learning classifier (8) using both the before and after datasets as prediction sets for the model to label. In brief, this random forest was constructed using a dataset composed of 4 classes (control individuals, mild-moderate cases, severe and PASC individuals). Because of the unbalanced nature of the dataset, the training set was subjected to a minority class balancing method that generates synthetic samples by means of interpolation (SMOTE) (23). Prediction of the labels was done once the model was fine-tuned, using an unseen test set, which was not subjected to SMOTE to avoid contamination or overfitting. We used this model to predict the labels of both groups in order to further validate the classification/labeling of the dataset individuals as PASC.

### D. Results

### 1. Comparison Between “Before” and “After” Treatment Demonstrates Statistical Differences Between Groups

The statistical comparison using the Wilcoxon paired test to contrast the before and after treatment groups using a two-sided alternative hypothesis revealed the existence of statistically significant differences between the cytokine profiles (biomarkers) between the before and after treatment groups (p-value=2.20e−17). For this test, group 1 was before and group 2 was after. The results of the Wilcoxon test support that the medians of both groups are different and that a one-tailed test needed to be done. Based on the results of the two-sided test, we proceeded to do a one-sided. The alternative hypothesis of this second test was focused on determining if the medians values for the biomarkers in treatment group 1 (before) were greater than those of group 2 (after). This test resulted in a statistically significant difference, where the p-value was less than the threshold of 0.05 (p-value=1.10e−17).

Our results indicate that the biomarker (cytokine profiles) of the individuals from individuals in the dataset before treatment are, statistically different from those after treatment. Moreover, our statistical test suggests that for these individuals, these biomarkers are statistically greater before treatment.

### 2. Correlation Analysis Indicates the Presence of Positive Correlations Between Cytokine Biomarkers and Subjective Scores

We constructed a correlation matrix using the Pearson's correlation coefficient in order to identify the positive correlations between the different biomarkers (cytokines) in the dataset and the subjective scores present. We calculated three correlation coefficients. The first is the joint correlation, which represents the relationship between the full dataset (both before and after treatment groups), followed by the coefficients for each treatment group, as shown in FIG. 1 (correlation matrix). In addition to the correlation coefficient, we calculated the corresponding p-value to support the statistical significance of these relationships. We defined our significance threshold to p-values of ≤0.05.

We analyzed the linear relationship between the cytokine biomarkers and the modified Rankin score (24). In brief, this is a 6-point disability scale that ranges from 0 (individual has no residual symptoms) to 5 (the individual is bedridden, incontinent and requires continuous care). According to the documentation an additional value of 6 is included for deceased or “expired” individuals. For the Rankin subjective score, we identified a low positive correlation with statistical significance for two biomarkers, VEGF and sCD40L (FIG. 2). Finally, we did the correlation analysis for the COMPASS 31 score (25). This scale was developed as a robust statistical instrument to determine autonomic symptoms, thus providing relevant severity scores for clinical assessment. For this scale, we identified that several cytokines had statistically significant relationships to the subjective score. TNF-alpha and GM CSF had low positive correlations, while VEGF and sCD40L showed moderate positive correlation (FIG. 3).

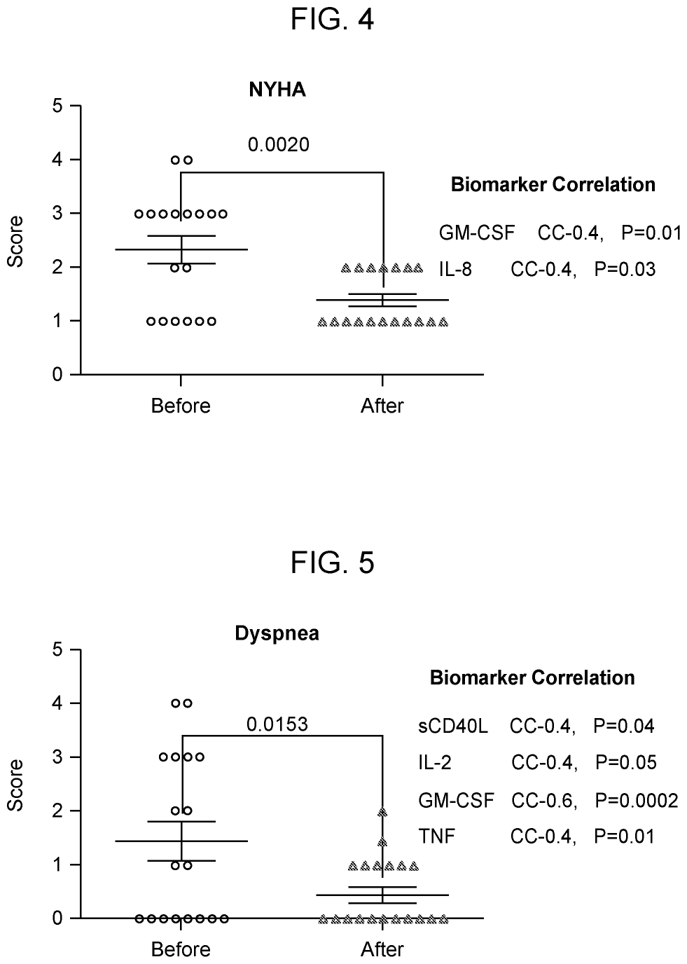

For it is possible to note that for the first subjective score, the New York Heart Association (NYHA) Functional Classification, which labels individuals in one of four categories, we were able to identify two statistically significant biomarkers in the joint correlation (FIG. 4). The cytokines IL-8 and GM-CSF showed a low positive correlation to the NYHA score, with both having rvalues between 0.30 and 0.50. The linear association between IL-8 and GM-CSF indicates that there appears to be a weak linear association between both treatment groups (before and after) where the levels of both cytokines appear to be positively associated with the NYHA score.

When subsequently analyzed the correlation values for the Medical Research Council (MRC) Dyspnea scale score (FIG. 5), which is a simple scale allowing participants to indicate the effects of breathlessness on mobility, we were able to identify that for both treatment groups (joint correlation), the biomarkers GM-CSF, TNF-alpha and sCD40L presented statistically correlations. In the case of GM-CSF, the linear association between the cytokine and the subjective score was 0.593, which makes it a moderate positive correlation. For TNF-alpha and sCD40L there correlation values were in ranges between 0.30 and 0.50, indicating their association with the MRC Dyspnea score were low positive.

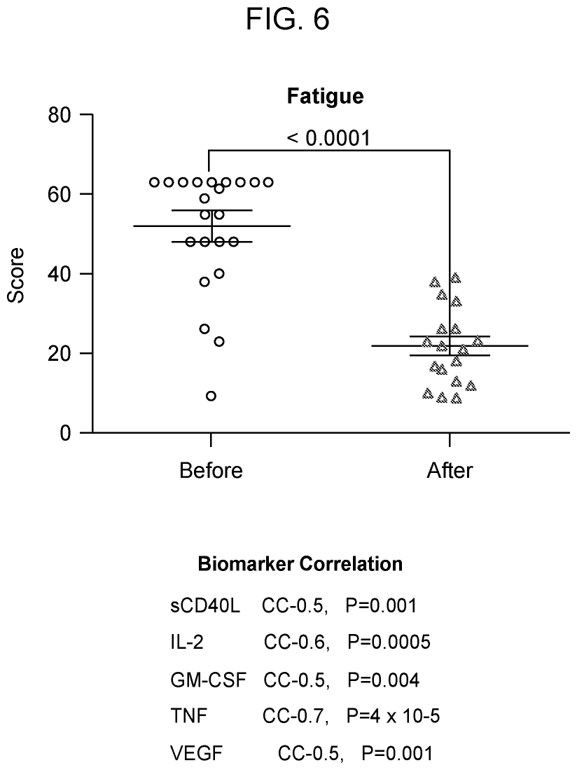

In addition, the correlation analysis of the Fatigue score from the Shirley Ryan Ability Lab at the Rehabilitation Institute of Chicago (https://www.sralab.org/rehabilitation-measures/fatigue-severity-scale) provides a 9-item scale allowing the measurement of the effects of fatigue on an individual. The scores range from a value of 9 (lowest possible score) to 63 (highest fatigue effects). Our analysis identified that various biomarkers showed statistically significant correlations (FIG. 6). These linear associations were present in both the before and after treatment groups (joint correlation). The cytokines IL-2, sCD40L, TNF alpha and VEGF presented a positive correlation, with r values ranging between 0.50 and 0.70, as shown in FIG. 1. In addition to these biomarkers, IL-8, IL-10 and GM CSF presented low positive correlations, with r values ranging between 0.30 and 0.50.

Our results indicate that there are a number of biomarkers that appear to be positively associated in varying degrees with the various subjective scores. The most common cytokine was sCD40L, positively associated to all scores except for the NYHA Functional Classification score. Another interesting finding is the relationship of GM-CSF to a wide variety of subjective scores. This cytokine had significant positive association to all scales except for the modified Rankin score. Finally, both VEGF and TNF-alpha were correlated with 3 of the 5 subjective scores, with VEGF not having a significant relation to NHYA and MRC Dyspnea, while TNF-alpha not correlating to NYHA and Rankin. These results suggest that many cytokine biomarkers possess for both the before and after treatment groups positive levels of statistically significant relationship.

### 3. Machine Learning Classifier Validates the Labelling of Individuals in the Dataset Group as PASC Using Cytokine Profiles

The individuals in the dataset were identified as being composed of long hauler or PASC individuals. In order to validate this assessment, we used the previously published random forest classifier (6) to label each of the treatment groups. The 36 instances (18 individuals for each treatment) were identified as belonging to the PASC class, according to the model. This classification was of great importance because it allowed us to use the long hauler/PASC heuristic score previously developed in (6) to further understand how these individuals have altered their behavior before and after treatment.

### E. Discussion

The discovery of CD16+ monocytes containing persistent S1 proteins from PASC patients may help further understand its pathophysiology and identify targets for therapy (4). Both CD16+ monocytes subsets, intermediate (CD14+CD16+) and nonclassical (CD14− CD16+), respectively, are known to interact significantly with the endothelium and platelets via the fractalkine pathway (26). This suggests that the pathophysiology of PASC may lie with the monocytic-endothelial-platelet axis. Fractalkine, which mediates cell adhesion and leucocyte recruitment, is a transmembrane protein expressed in the brain, colon, heart, and lung, along with endothelial cells and astrocytes. Intermediate monocytes express high levels of both CCR5 and fractalkine receptors, whereas nonclassical monocytes express high levels of fractalkine receptors (6,7). This interaction between fractalkine and fractalkine receptors have been involved in the pathogenesis of atherosclerosis, vasculitis, vasculopathies, and inflammatory brain disorders (27) and could also be contributing to vascular endothelialitis in PASC. Vascular endothelialitis leads to collagen exposure along with platelet activation and adherence via glycoprotein 1b-IX-V-receptor (GPlb-IX-V) with collagen-bound von Willebrand factor (vWF) (28). Activated platelets release soluble CD40 ligand (sCD40L) to recruit both neutrophils and monocytes to the vascular lesions (29), thus activating the coagulation cascade. Stimulated platelets also release RANTES which binds to endothelial cells and encourages monocyte adhesion to inflamed endothelial tissues (30) and acts as a chemotactic agent for inflammatory cells. Activated platelets and endothelial cells can also secrete VEGF which induces angiogenesis and microvascular hyperpermeability. VEGF is a diagnostic marker for vasculitic neuropathy and also contributes to a pro-inflammatory-prothrombotic environment (31). While the vascular effects of statins have been well documented (32), the protective role of maraviroc on the endothelium has also been similarly published (33). Hence, we targeted CCR5 and fractalkine receptors on the S1 protein expressing CD16+ monocytes using maraviroc and pravastatin, respectively, hypothesizing that this combination could be therapeutically effective in treating vascular endothelialitis and resolving symptoms associated with PASC.

Neurological symptoms associated with PASC include severe headaches and cognitive impairment (brain fog), along with neuropathy and weakness, necessitating the need for assistance in performing daily tasks. CD14+CD16+ monocytes are known to transmigrate across the blood brain barrier and play an important role in central nervous system (CNS) immune surveillance. These monocytes were implicated as HIV reservoirs in the CNS causing neuroinflammation, neuronal damage, and cognitive defects (34). We hypothesize that the S1 protein containing CD14+CD16+ monocytes in PASC patients are also crossing the blood brain barrier and triggering neuroinflammation and inducing neurological symptoms. Both maraviroc and statins are known to cross the blood-brain-barrier, and more specifically, maraviroc has been suggested as treatment for Parkinson's, neurocognitive impairment, and strokes (35). Interestingly, after the introduction of maraviroc and pravastatin, participants showed a decrease in modified Rankin scale scores (p=0.0002) (FIG. 2) and reported improvement in neurological function and ability. These findings were correlated with a statistically significant decrease in VEGF (r=0.4, p=0.02) and sCD40L (r=0.42, p=0.01), suggesting treatment targeting cytokines associated with vascular endothelialitis correlated with improvement in neurological symptoms.

Autonomic dysfunction such as postural orthostatic tachycardia syndrome (POTS) and light sensitivity has also been associated with PASC. POTS is a syndrome consisting of unexplained tachycardia, dizziness, light-headedness, fainting, and abdominal pain. While the true etiology of POTS has yet to be defined, endothelial dysfunction has been suggested as the pathophysiology (36). There is also evidence that POTS maybe be associated with G-protein-coupled receptor autoantibodies (37). Interestingly, since CCR5 and fractalkine receptor are also G-protein-coupled receptors (9,38), it is possible that antagonism of these receptors could also inhibit the autonomic effects of these autoantibodies. We observed a statistically significant decrease in COMPASS-31 (p=0.0001) (FIG. 3) scores correlating with statistically significant decreases in VEGF (r=0.6, p=0.0005), sCD40L (r=0.6, p=0.0001), and TNF-alpha (r=0.5, p=0.0026), suggesting that pro-inflammatory macrophage activation may be triggering vascular endothelialitis. Interestingly, elevations in sCD40L have also been associated with sympathoadrenal activation and targeting these vascular markers may address PASC associated dysautonomia (39).

Cardiorespiratory complaints such as chest pain, shortness of breath, and symptoms resembling POTS are very commonly reported by PASC patients. Many PASC patients with cardiac and pulmonary symptoms have undergone extensive workup (EKG, echocardiogram, stress test, pulmonary function testing, etc.) which have not detected any abnormalities or pathologies. Subsequently, current clinical approaches have only been used to treat symptoms with antiarrhythmics, bronchodilators or alpha-adrenergics, instead of addressing the underlying pathophysiology. We observed an improvement in cardiac symptoms evidenced by a decrease in NYHA functional classification (p=0.002) (FIG. 4). This improvement was associated with statistically significant decreases in IL-8 (r=0.4, p=0.03) and GM-CSF (r=0.4, p=0.01). Interestingly, endothelial cells are main producers of IL-8 (40) and statins are known to decrease IL-8 (41). Additionally, maraviroc has been suggested as reducing the cardiovascular risk for acute coronary disease by protecting the endothelium from pro-inflammatory macrophage infiltration (42). These mechanisms potentially support their use in addressing PASC associated cardiac symptoms. We also observed improvement in respiratory symptoms after initiating maraviroc and pravastatin therapy. Participants reported improvements as reflected by a statistically significant decrease in the MRC Dyspnea scale (p=0.0153) (FIG. 5). These responses and improvements correlated with statistically significant decreases in IL-2 (r=0.4, p=0.05), GM-CSF (r=0.6, p=0.0002), sCD40L (r=0.4, p=0.04), and TNF-alpha (r=0.4, p=0.01). Intriguingly, CD16+ monocytes are known to produce large quantities of TNF-alpha and could be activated by the retained S1 proteins (43), causing vascular endothelialitis via the fractalkine-fractalkine receptor interaction in pulmonary vasculature. Elevations in sCD40L have been associated with pulmonary arterial hypertension (PAH) (44), while IL-2 can induce pulmonary microvasculature injury and generate an asthma-like bronchoconstriction (45). We previously published a multi-class model score that described an increase IL-2 as a characteristic specific to PASC (8), thus confirming the clinical significance of IL-2 in PASC. Both maraviroc and statins can decrease IL-2 and TNF-alpha (41,46), which may explain the observed improvements in PASC associated respiratory symptoms. The patient Fatigue Severity Score (FSS) also significantly decreased (p<0.0001) (FIG. 6) after maraviroc and pravastatin which correlated with decrease in sCD40L (r=0.5, p=0.001), VEGF (r=0.5, p=0.001), TNF-alpha (r=0.7, p=4e−5), IL-2 (r=0.6, p=0.0005), and GM-CSF (r=0.5, p=0.004), again suggesting that targeting the monocytic-platelet-endothelial axis can alleviate PASC associated fatigue.

Despite a black box warning for hepatoxicity, maraviroc has demonstrated a strong safety profile in adult, pediatric, and neonatal populations (47,48). Analysis of the MOTIVATE study demonstrated a low incidence of hepatoxicity with maraviroc even after 96 weeks of treatment at the FDA approved dose of 300 mg B.I.D (49). This influenced our decision to treat with this dose. Hepatic safety was monitored in all the participants by measuring and evaluating AST, ALT, and total bilirubin (LFTs) prior to commencing treatment with maraviroc and every two weeks while on treatment. None of participants presented here experienced any clinical signs of hepatotoxicity or elevated liver function serologies while on, or after, treatment. Maraviroc is metabolized by CYP3A4, and we chose to avoid any CYP3A4 metabolizing statins to mitigate any potential drug interactions. This approach guided our decision to treat with pravastatin 10 mg PO daily over the other statins since it is metabolized via glucuronidation. However, the therapeutic benefits with other statins have also been observed and could be considered.

Since some of the participants were already on other therapeutics including ivermectin, fluvoxamine, and prednisone, all the biomarker data and subjective responses were documented from the initiation of maraviroc and pravastatin. Some participants saw symptom relief after six weeks and were ready to stop all medications, while others needed treatment up to twelve weeks before discontinuing medications.

### F. References

1. Soriano J B, Murthy S, Marshall J C, Relan P, Diaz J V, WHO Clinical Case Definition Working Group on Post-COVID-19 Condition. A clinical case definition of Post-COVID-19 condition by a Delphi consensus. Lancet Infect Dis. Published online Dec. 21, 2021: S1473-3099(21)00703-9.
2. Davis H E, Assaf G S, McCorkell L, et al. Characterizing Long COVID in an International Cohort: 7 months of symptoms and their impact. eClinicalMedicine. 2021; 38.
3. Novak P, Mukerji S S, Alabsi H S, et al. Multisystem Involvement in Post-Acute Sequelae of Coronavirus Disease 19. Ann Neurol. Published online Dec. 24, 2021.
4. Patterson B K, Francisco E B, Yogendra R, et al. Persistence of SARS-CoV2-2 S1 Protein in CD16+ Monocytes in Post-Acute Sequelae of Covid-19 (PASC) up to 15 months post-infection. Front Immunol. 2022; 0.
5. Thomas G, Tacke R, Hedrick C C, Hanna R N. Nonclassical Patrolling Monocyte Function in the Vasculature. Arteriosclerosis, Thrombosis, and Vascular Biology. 2015; 35(6):1306-1316.
6. Kapellos T S, Bonaguro L, Gemund I, et al. Human Monocyte Subsets and Phenotypes in Major Chronic Inflammatory Diseases. Front Immunol. 2019; 0.
7. Weber C, Belge K U, von Hundelshausen P, et al. Differential Chemokine Receptor Expression and Function in Human Monocyte Subpopulations. J Leukoc Biol. 2000; 67(5):699-704.
8. Patterson B K, Guevara-Coto J, Yogendra R, et al. Immune-based Prediction of COVID-19 Severity and Chronicity Decoded Using Machine Learning. Front Immunol. 2021; 0.
9. Oppermann M. Chemokine receptor CCR5: insights into Structure, Function, and Regulation. Cell Signal. 2004; 16(11):1201-1210.
10. Vangelista L, Vento S. The Expanding Therapeutic Perspective of CCR5 Blockade. Front Immunol. 2018; 0.
11. Marques R E, Guabiraba R, Russo R C, Teixeira M M. Targeting CCL5 in Inflammation. Expert Opin Ther Targets. 2013; 17(12):1439-1460.
12. Patterson B K, Seethamraju H, Dhody K, et al. CCR5 Inhibition in Critical COVID-19 Patients Decreases Inflammatory Cytokines, Increases CD8 T-cells, and Decreases SARS-CoV2 RNA in Plasma by Day 14. International Journal of Infectious Diseases. 2021; 103:25-32.
13. Bowen G P, Borgland S L, Lam M, Libermann T A, Wong N C W, Muruve D A. Adenovirus Vector-induced Inflammation: Capsid-Dependent Induction of the C-C chemokine RANTES requires NF-kappa B. Hum Gene Ther. 2002; 13(3):367-379.
14. Arberas H, Guardo A C, Bargalló M E, et al. In Vitro Effects of the CCR5 Inhibitor Maraviroc on Human T cell Function. J Antimicrob Chemother. 2013; 68(3):577-586.
15. Pozo-Balado M M, Rosado-Sánchez I, Mendez-Lagares G, et al. Maraviroc Contributes to the Restoration of the Homeostasis of Regulatory T-cell Subsets in Antiretroviral-Naive HIV-Infected Subjects. Clin Microbiol Infect. 2016; 22(5):461.e1-5.
16. Cimato T R, Palka B A. Fractalkine (CX3CL1), GM-CSF and VEGF-A Levels are Reduced by Statins in Adult Patients. Clin Transl Med. 2014; 3:14.
17. Eisinger F, Patzelt J, Langer H F. The Platelet Response to Tissue Injury. Front Med. 2018; 0.
18. Model MS-JMG, 1999 undefined. Python: a programming language for software integration and development. academia.edu Available at: https://www.academia.edu/download/25505223/10.1.1.35.6459.pdf [Accessed Feb. 3, 2022]
19. Mckinney W. pandas: a Foundational Python Library for Data Analysis and Statistics. Available at: http://pandas.sf.net [Accessed Apr. 17, 2021]
20. Virtanen P, Gommers R, Oliphant T E, Haberland M, Reddy T, Cournapeau D, Burovski E, Peterson P, Weckesser W, Bright J, et al. SciPy 1.0: fundamental algorithms for scientific computing in Python. Nat Methods 2020 173 (2020) 17:261-272. doi:10.1038/s41592-019-0686-2
21. Ihaka R, Gentleman R. R: A Language for Data Analysis and Graphics. J Comput Graph Stat (1996) 5:299-314. doi:10.1080/10618600.1996.10474713
22. Emerson J W, Green W A, Schloerke B, Crowley J, Cook D, Hofmann H, Wickham H. The generalized pairs plot. J Comput Graph Stat (2013) 22:79-91.
23. Chawla N V., Bowyer K W, Hall L O, Kegelmeyer W P. SMOTE: Synthetic minority over-sampling technique. J Artif Intell Res (2002) 16:321-357. doi:10.1613/jair.953
24. Modified Rankin Score (mRS). Available at: https://manual.jointcommission.org/releases/TJC2018A/DataElem0569.html [Accessed Feb. 3, 2022]
25. Sletten D M, Suarez G A, Low P A, Mandrekar J, Singer W. COMPASS 31: a refined and abbreviated Composite Autonomic Symptom Score. Mayo Clin Proc (2012) 87:1196-1201. doi:10.1016/J.MAYOCP.2012.10.013
26. Kasama T, Wakabayashi K, Sato M, Takahashi R, Isozaki T. Relevance of the CX3CL1/Fractalkine-CX3CR1 Pathway in Vasculitis and Vasculopathy. Transl Res. 2010; 155 (1):20-26.
27. Thomas G, Tacke R, Hedrick C C, Hanna R N. Nonclassical Patrolling Monocyte Function in the Vasculature. Arteriosclerosis, Thrombosis, and Vascular Biology. 2015; 35(6):1306-1316.
28. Varga-Szabo D, Pleines I, Nieswandt B. Cell Adhesion Mechanisms in Platelets. Arteriosclerosis, Thrombosis, and Vascular Biology. 2008; 28 (3):403-412.
29. Flierl U, Bauersachs J, Schafer A. Modulation of Platelet and Monocyte Function by the Chemokine Fractalkine (CX3CL1) in Cardiovascular Disease. Eur J Clin Invest. 2015; 45(6):624-633.
30. von Hundelshausen P, Weber K S, Huo Y, et al. RANTES Deposition by Platelets Triggers Monocyte Arrest on Inflamed and Atherosclerotic Endothelium. Circulation. 2001; 103(13):1772-1777.
31. Cuadrado M J, Buendía P, Velasco F, et al. Vascular Endothelial Growth Factor Expression in Monocytes from Patients with Primary Antiphospholipid Syndrome. J Thromb Haemost. 2006; 4 (11):2461-2469.
32. Liberale L, Carbone F, Montecucco F, Sahebkar A. Statins reduce vascular inflammation in atherogenesis: A review of underlying molecular mechanisms. Int J Biochem Cell Biol. 2020 May; 122:105735. doi:10.1016/j.biocel.2020.105735. Epub 2020 Feb. 29. PMID: 32126319.
33. Francisci D, Pirro M, Schiaroli E, et al. Maraviroc Intensification Modulates Atherosclerotic Progression in HIV-Suppressed Patients at High Cardiovascular Risk. A Randomized, Crossover Pilot Study. Open Forum Infect Dis. 2019; 6 (4):ofz112.
34. Veenstra M, Leon-Rivera R, Li M, Gama L, Clements J E, Berman J W. Mechanisms of CNS Viral Seeding by HIV+CD14+CD16+ monocytes: Establishment and Reseeding of Viral Reservoirs Contributing to HIV-Associated Neurocognitive Disorders. mBio. Published online Oct. 24, 2017.
35. Mondal S, Rangasamy S B, Roy A, Dasarathy S, Kordower J H, Pahan K. Low-dose Maraviroc, an Antiretroviral Drug, Attenuates the Infiltration of T-cells into the Central Nervous System and Protects the Nigrostriatum in Hemiparkinsonian Monkeys. The Journal of Immunology. 2019; 202(12):3412-3422.
36. Liao Y, Chen S, Liu X, et al. Flow-Mediated Vasodilation and Endothelium Function in Children with Postural Orthostatic Tachycardia Syndrome. Am J Cardiol. 2010; 106(3):378-382.
37. Gunning W T, Stepkowski S M, Kramer P M, Karabin B L, Grubb B P. Inflammatory Biomarkers in Postural Orthostatic Tachycardia Syndrome with Elevated G-Protein-Coupled Receptor Autoantibodies. J Clin Med. 2021; 10(4):623.
38. Poniatowski T A, Wojdasiewicz P, Krawczyk M, et al. Analysis of the Role of CX3CL2 (Fractalkine) and its Receptor CX3CR1 in Traumatic Brain and Spinal Cord Injury: Insight into Recent Advances in Actions of Neurochemokine Agents. Mol Neurobiol. 2017; 54(3):2167-2188.
39. Johansson P I, Sørensen A M, Perner A, et al. High sCD40L Levels Early After Trauma are Associated with Enhanced Shock, Sympathoadrenal Activation, Tissue and Endothelial Damage, Coagulopathy and Mortality. J Thromb Haemost. 2012; 10 (2):207-216.
40. Oude Nijhuis C S M, Vellenga E, Daenen S M G J, Kamps W A, De Bont E S J M. Endothelial Cells Are Main Producers of Interleukin 8 through Toll-like Receptor 2 and 4 Signaling During Bacterial Infection in Leukopenic Cancer Patients. Clin Diagn Lab Immunol. 2003; 10(4):558-563.
41. Sakoda K, Yamamoto M, Negishi Y, Liao J K, Node K, Izumi Y. Simvastatin Decreases IL-6 and IL-8 Production in Epithelial Cells. J Dent Res. 2006; 85(6):520-523.
42. Cipriani S, Francisci D, Mencarelli A, et al. Efficacy of the CCR5 Antagonist Maraviroc in Reducing Early, Ritonavir-Induced Atherogenesis and Advanced Plaque Progression in Mice. Circulation. 2013; 127 (21):2114-2124.
43. Belge K U, Dayyani F, Horelt A, et al. The proinflammatory CD14+CD16+DR++ monocytes are a major Source of TNF. The Journal of Immunology. 2002; 168(7):3536-3542.
44. Dam\dot{a}s J K, Otterdal K, Yndestad A, et al. Soluble CD40 Ligand in Pulmonary Arterial Hypertension. Circulation. 2004; 110(8):999-1005.
45. Rabinovici R, Sofronski M D, Borboroglu P, et al. Interleukin-2-Induced Lung Injury. The Role of Complement. Circ Res. 1994; 74(2):329-335.
46. Kim S W, Kang H J, Jhon M, et al. Statins and Inflammation: New Therapeutic Opportunities in Psychiatry. Front Psychiatry. 2019; 0.
47. Giaquinto C, Mawela M P, Chokephaibulkit K, et al. Pharmacokinetics, Safety and Efficacy of Maraviroc in Treatment-Experienced Pediatric Patients Infected with CCR5-Tropic HIV-1. Pediatr Infect Dis J. 2018; 37(5):459-465.
48. Gulick R M, Fatkenheuer G, Burnside R, et al. Five-year Safety Evaluation of Maraviroc in HIV-1—infected Treatment-Experienced Patients. J Acquir Immune Defic Syndr. 2014; 65(1):78-81.
49. Ayoub, Aymana; Alston, Sama; Goodrich, Jamesb; Heera, Jayvantb; Hoepelman, Andy IMc; Lalezari, Jacobd; Mchale, Marya; Nelson, Marke; van der Ryst, Elnaa; Mayer, Howardf Hepatic Safety and Tolerability in the Maraviroc Clinical Development Program, AIDS: Nov. 13, 2010—Volume 24—Issue 17—p 2743-2750

### III. Cytokine Hub Classification of PASC, ME-CFS and Other PASC-Like Conditions

### A. Abstract

Post-acute sequelae of COVID-19 (PASC) is a growing healthcare and economic concern affecting as many as 10%-30% of those infected with COVID-19. Though the symptoms have been well-documented, they significantly overlap with other common chronic inflammatory conditions which could confound treatment and therapeutic trials.

A total of 236 patients including 64 with post-acute sequelae of COVID-19 (PASC), 50 with myalgic encephalomyelitis-chronic fatigue syndrome (ME-CFS), 29 with post-treatment Lyme disease (PTLD), and 42 post-vaccine individuals with PASC-like symptoms (POVIP) were enrolled in the study. We performed a 14-plex cytokine/chemokine panel previously described to generate raw data that was normalized and run in a decision tree model using a Classification and Regression Tree (CART) algorithm. The algorithm was used to classify these conditions in distinct groups despite their similar symptoms. PASC, ME-CSF, POVIP, and Acute COVID-19 disease categories were able to be classified by our cytokine hub based CART algorithm with an average F1 score of 0.61 and high specificity (94%). Proper classification of these inflammatory conditions with very similar symptoms is critical for proper diagnosis and treatment.

### B. Introduction

Investigation has identified overlapping symptom presentations of PASC with ME/CFS, PTLD and other post-infectious chronic inflammatory disorders (Wong & Weitzer, “Long COVID and Myalgic Encephalomyelitis/Chronic Fatigue Syndrome (ME/CFS)-A Systemic Review and Comparison of Clinical Presentation and Symptomatology,” Medicina (Kaunas). 2021 Apr. 26; 57 (5):418; Patterson et. al., “Immune-based prediction of COVID-19 severity and chronicity decoded using machine learning,” Front Immunol. 2021 Jun. 28; 12:700782; Wong et al., “A Review of Post-treatment Lyme Disease Syndrome and Chronic Lyme Disease for the Practicing Immunologist,” Clin Rev Allergy Immunol. 2022 February; 62 (1):264-271). However, clear etiological and pathophysiological differences exist in these conditions that necessitate precision medicine tailored therapies. A recent report suggested the use of cytokine hubs to more precisely categorize autoimmune diseases with the stated goal of using the information as therapeutic targets as the expansion of immune based therapy grows (Schett et al., “Reframing Immune-Mediated Inflammatory Diseases through Signature Cytokine Hubs,” N Engl J Med. 2021; 385 (7):628-639). The heterogeneity of immune-mediated inflammatory diseases (IMIDS) described in the prior publication also applies to post-infectious immune-mediated and inflammatory conditions currently in the discussion of post-infectious sequelae of COVID-19 (PASC) and the focus of this report. Unlike the previous publication, PASC-like conditions share many of the same symptoms making diagnosis and, ultimately, treatment more difficult. Here, we present a machine learning approach to classifying these symptom related conditions.

### C. Methods

### 1. Patients

After written informed consent was obtained, the immunological lab results were used for the current analysis of 236 patients including 64 with post-acute sequelae of COVID-19 (PASC), 26 with mildmoderate acute COVID-19 (MM), 25 with severe acute COVID-19, 50 with myalgic encephalomyelitischronic fatigue syndrome (ME-CFS), 29 with post-treatment Lyme disease (PTLD), and 42 post-vaccine individuals with PASC-like symptoms (POVIP) defined as COVID-negative (nucleoprotein Ab negative, Tcell immunity negative) individuals with PASC-like symptoms 3 months after the last vaccination.

### 2. Multiplex Cytokine Quantification

Fresh plasma was used quantification of the following analytes: TNF-α, IL-4, IL-13, IL-2, GM-CSF, sCD40L, CCL5 (RANTES), CCL3 (MIP-1α), IL-6, IL-10, IFN-γ, VEGF, IL-8, and CCL4 (MIP-1β) as previously described (Patterson et al., Id).

### 3. Data Acquisition and Processing

The data [Mild-Moderate acute COVID, Severe acute COVID, PASC, POVIP, ME-CSF and PTLD individuals] consisted of a total of 16 columns represented by an anonymized sample identifier, and the last column was the class or disease state assigned to the individual. The class label was removed from this pandas dataset and assigned into a new variable (target) (Mckinney W., “Pandas: A Foundational Python Library for Data Analysis and Statistics”, http://pandas.sf.net).

The dataset comprising the cytokine profiles was normalized using L2 (Euclidian) normalization. The L2 nonrealization approach calculates the distance of a given vector of values, such as the cytokine values for each data point or instance from the origin of the vector space. The implementation of the L2 norm, which is a positive value, can be supported by the notion our model is focused on identifying the differences between classes, thus the signal or pattern that characterizes each class and not the effect of the magnitudes on each class (Brownlee, “Basics of Linear Algebra for Machine Learning: Discover the Mathematical Language of Data in Python”, 2018). The two datasets (cytokines profiles and targets/labels) were used to train a machine learning classifier using decision trees to define patient's disease state (Breiman et al., “Classification and regression trees,” Classif Regres Trees. Published online Jan. 1, 2017:1-358).

### 4. Multiclass Classification of Disease States Using a Decision Tree Model

The decision tree model was based on the Classification and Regression Trees (CART) algorithm in scikitlearn (Pedregosa et al., “Scikit-Learn: Machine Learning in Python,” Vol 12; 2011. Accessed Apr. 17, 2021. http://scikit-learn.sourceforge.net). The model was fine-tuned using GridSearchCV from scikit-learn. To fine-tune the model, we selected the following hyperparameters: maximum number of features, the minimum number of samples to split a node, the minimum number of samples per node, the maximum depth of the tree. Additionally, the impurity criterion was defined as the Gini impurity value. The cross validation was set to 10-fold cross validation with 3 repeats.

The decision tree was used to calculate a confusion matrix in order to visualize the model's predictive power as well as a “leave one out cross-validation” (LOOCV). The confusion matrix was used to calculate the F1-score which uses the harmonic means of precision and recall; combining them into a performance metric ranging from 0 (poor) to 1 (perfect). The resulting tree was then plotted to visualize the separation of the classes based on the different cytokines.

### D. Results And Discussion

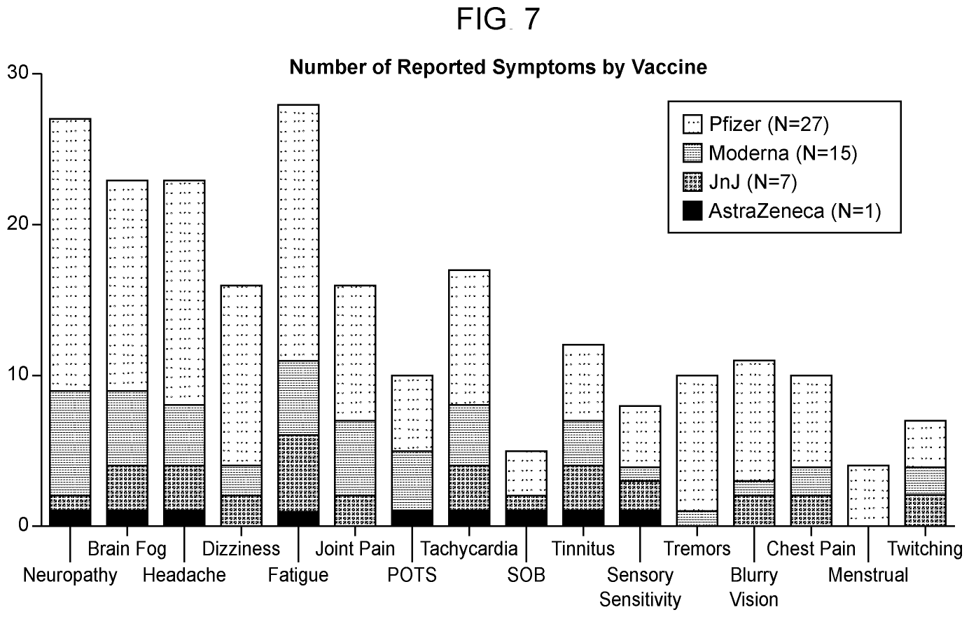

The symptoms of PASC have been widely reported and significantly overlap with ME-CFS and with PTLD. In addition, we included patients, of unknown prevalence, who are post-vaccine individuals with PASC-like symptoms (POVIP) in the present study (FIG. 7). These patients experience PASC-like symptoms 3 months minimum post-vaccination in the absence of COVID 19 infection.

By using a decision tree classifier, Classification and Regression Trees (CART), we developed an algorithm to propose a simple but powerful predictive model with high interpretability, a characteristic of great importance when attempting to understand differences between disease states. Our model had an average weighted F1 score of 0.61, which was variable due to the stochastic nature of both the model and the dataset.

TABLE 1

| Class | Recall | Precision | Specificity | F1 Score |
|---|---|---|---|---|
| MM | 0.8846 | 0.7419 | 0.9619 | 0.8070 |
| Severe | 0.6000 | 0.7143 | 0.9716 | 0.6522 |
| PASC | 0.8438 | 0.8308 | 0.9360 | 0.8372 |
| Post-Vax | 0.8333 | 0.6140 | 0.8866 | 0.7071 |
| ME-CSF | 0.7000 | 0.6604 | 0.9032 | 0.6796 |
| Lyme | 0.1724 | 0.5556 | 0.9807 | 0.2632 |
| Average | 0.6724 | 0.6862 | 0.9400 | 0.6577 |

As shown in Table 1 and in the confusion matrix (Supplementary FIG. 1), the model was robust in its ability to identify four of the six classes of disease states (e.g., MM, Severe, PASC, and ME-CSF). Some misclassification was demonstrated in the remaining two classes (POVIP and Lyme) that was likely due to overlapping cytokine hubs. The confusion matrix may suggest that the immune contribution to these two states were similar. Clinical data as well as other immunological parameters could potentially separate these two conditions and further increase the model's predictive power.

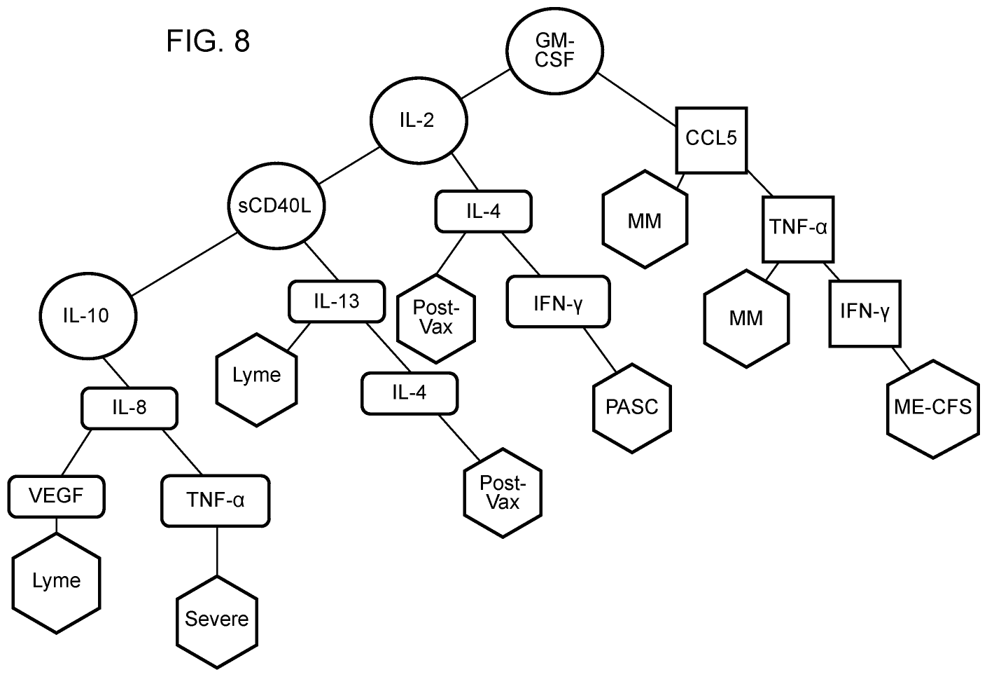

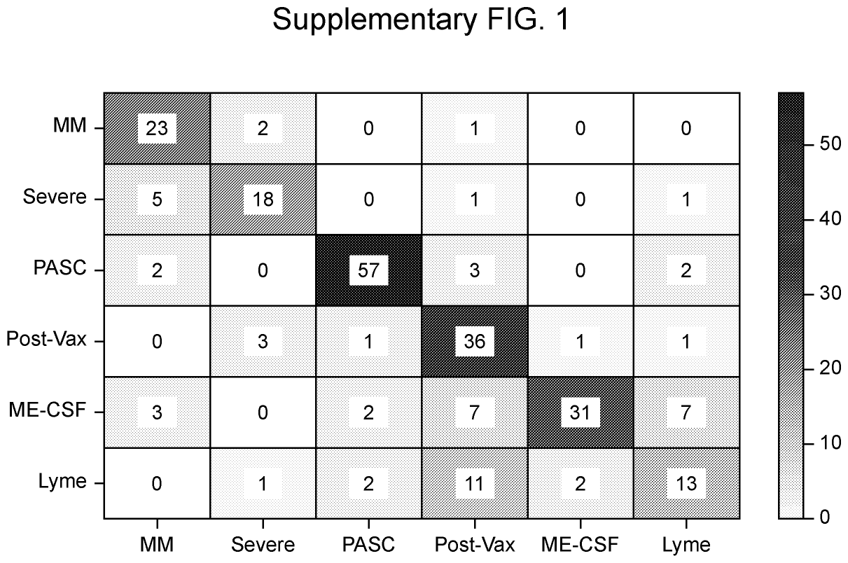

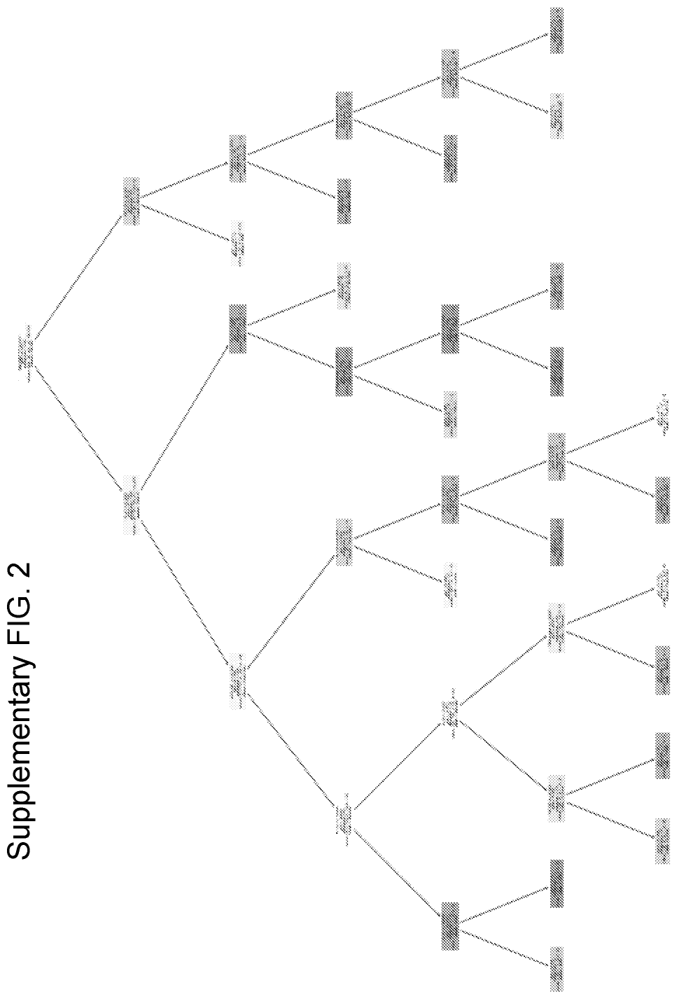

The highest performance metrics after fine-tuning was provided by the CART decision tree (FIG. 8) when data were plotted using internal tree plotting functions and python's matplotlib (Supplementary FIG. 2). As shown in the plotted tree (FIG. 8, Supplementary FIG. 2), we demonstrate that the CART algorithm was capable of constructing splitting and terminal hubs with low Gini impurity values and high F1 scores for those classes shown previously in the confusion matrix (Supplementary FIG. 1). In the POVID and PTLD classes, splitting hubs with higher Gini impurity values were observed. The identification of these hubs supports the possibility that the immune profiles of both POVIP and PTLD individuals have similar cytokine patterns.

The disease heterogeneity suggests that the classification need not perfectly match the labels and that individuals within each of these conditions might present different immunological entities potentially requiring differential therapeutics. We demonstrated that for POVIP and MM, three or more distinct cytokine profiles might be important for their classification supporting the presence of different immunological entities within these groups. On the other hand, for ME-CSF, Lyme, and PASC only one or two cytokine classification profiles were found.

The PASC classification highlights the importance of the proinflammatory cytokines IL-2 and IFN-γ as we have previously reported (Patterson et al., Id.), while in PTLD disease, two classification profiles were identified. Interestingly, both profiles follow a common path including the proinflammatory cytokines GM-CSF and IL-2 in concordance with IL-2 mediated GM-CSF production previously reported (Hartmann et al., “Multiple Sclerosis-Associated IL2RA Polymorphism Controls GM-CSF Production in Human TH Cells,” Nat Commun. 2014 Oct. 3; 5:5056). One PTLD profile appears to be driven by IL-8 induced responses while the other mediated by IL-13. PTLD includes a variety of clinical features and pathogenic mechanisms with two different immune clusters (Steere A C, “Posttreatment Lyme Disease Syndromes: Distinct Pathogenesis Caused by Maladaptive Host Responses” J Clin Invest. 2020 May 1; 130 (5):2148-2151). Both share features that include T cell receptor signaling and involvement of monocytes/CD4+ T cells. The first cluster characterized by a type 1 inflammatory response associated with post-infectious Lyme arthritis and autoimmune joint disease that are associated with IL-8 (Steer, Id.; Clarke et al., “Predicting Lyme Disease From Patients' Peripheral Blood Mononuclear Cells Profiled With RNASequencing,” Front Immunol. 2021 Mar. 8). The second cluster includes activation of neutrophils and IL-4/IL-13 signaling (Clarke et al., Id.) which aligns more with post-treatment neurological disease (Steere, Id.). These data could explain the two different classification profiles, reported in this study, associated with different clinical manifestations of PTLD.

### E. Conclusions

We agree with the conclusions of Schett et al., that targeting of individual cytokines underlying the immunopathogenesis of these conditions may provide a powerful new tool in the treatment of these immunologically-mediated disorders using precision medicine.

Notwithstanding the appended claims, the disclosure is also defined by the following clauses:

1. A method of treating a subject for long-haul COVID, the method comprising:
administering to the subject a tropane CCR5/CCL5 interaction inhibitor in combination with a statin to treat the subject for long-haul COVID.
   2. The method according to Clause 1, wherein the tropane CCR5/CCL5 interaction inhibitor is described by the formula:

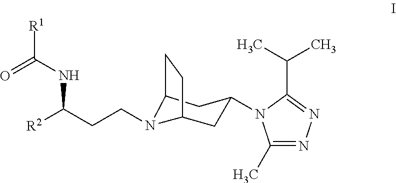

or a pharmaceutically acceptable salt or solvate thereof,
   wherein:
R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms.
   3. The method according to Clause 2, wherein the tropane CCR5/CCL5 interaction inhibitor is described by the formula:

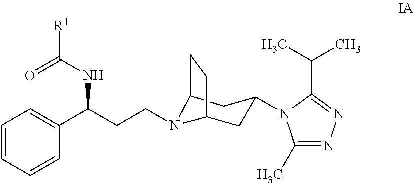

or a pharmaceutically acceptable salt or solvate thereof, wherein:
   R¹ is either C₃₋₆ cycloalkyl optionally substituted by one or more fluorine atoms, or C₁₋₆ alkyl optionally substituted by one or more fluorine atoms.
      4. The method according to Clause 3, wherein R¹ is either C₄₋₆ cycloalkyl optionally substituted by one or two fluorine atoms, or C₁₋₄ alkyl optionally substituted by from one to three fluorine atoms.
      5. The method according to Clause 4, wherein R¹ is either cyclobutyl, cyclopentyl, 4,4-difluorocyclohexyl or 3,3,3-trifluoropropyl.
      6. The method according to Clause 2, wherein R² is phenyl optionally substituted by 1 or 2 fluorine atoms.
      7. The method according to Clause 6, wherein R² is phenyl or monofluorophenyl.
      8. The method according to Clause 7, wherein R² is phenyl or 3-fluorophenyl.
      9. The method according to Clause 1, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl}cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide
or a pharmaceutically acceptable salt or solvate of any thereof.
   10. The method according to Clause 9, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).
   11. The method according to any of the preceding clauses, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.
   12. The method according to any of the preceding clauses, wherein the tropane CCR5/CCL5 interaction inhibitor and statin are administered sequentially to the subject.
   13. The method according to Clause 12, wherein the tropane CCR5/CCL5 interaction inhibitor is administered before the statin to the subject.
   14. The method according to Clause 12, wherein the tropane CCR5/CCL5 interaction inhibitor is administered after the statin to the subject.
   15. The method according to any of the preceding clauses, wherein the tropane CCR5/CCL5 interaction inhibitor and statin are administered simultaneously to the subject.
   16. The method according to Clause 15, wherein the tropane CCR5/CCL5 interaction inhibitor and statin are administered as separate pharmaceutical formulations to the subject.
   17. The method according to Clause 16, wherein the tropane CCR5/CCL5 interaction inhibitor and statin are administered in the same pharmaceutical formulation to the subject.
   18. The method according to Clause 17, wherein the pharmaceutical formulation comprises an oral formulation.
   19. The method according to Clause 18, wherein the oral formulation comprises a tablet.
   20. The method according to any of the preceding clauses, wherein the subject is a mammal.
   21. The method according to Clause 20, wherein the mammal is a human.
   22. A pharmaceutical composition comprising:
   a tropane CCR5/CCL5 interaction inhibitor; and
   a statin.
      23. The pharmaceutical composition according to Clause 22, wherein the tropane CCR5/CCL5 interaction inhibitor is described by the formula:

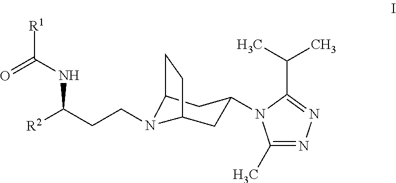

or a pharmaceutically acceptable salt or solvate thereof,
   wherein:
R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms.
   24. The pharmaceutical composition according to Clause 23, wherein the tropane CCR5/CCL5 interaction inhibitor is described by the formula:

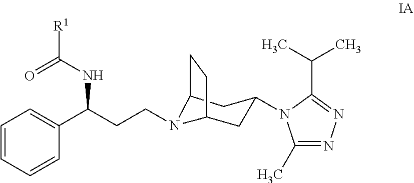

or a pharmaceutically acceptable salt or solvate thereof, wherein:
   R¹ is either C₃₋₆ cycloalkyl optionally substituted by one or more fluorine atoms, or C₁₋₆ alkyl optionally substituted by one or more fluorine atoms.
      25. The pharmaceutical composition according to Clause 24, wherein R¹ is either C₄₋₆ cycloalkyl optionally substituted by one or two fluorine atoms, or C₁₋₄ alkyl optionally substituted by from one to three fluorine atoms.
      26. The pharmaceutical composition according to Clause 25, wherein R¹ is either cyclobutyl, cyclopentyl, 4,4-difluorocyclohexyl or 3,3,3-trifluoropropyl.
      27 The pharmaceutical composition according to Clause 23, wherein R² is phenyl optionally substituted by 1 or 2 fluorine atoms.
      28. The pharmaceutical composition according to Clause 27, wherein R² is phenyl or monofluorophenyl.
      29. The pharmaceutical composition according to Clause 28, wherein R² is phenyl or 3-fluorophenyl.
      30. The pharmaceutical composition according to Clause 22, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl}cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide
or a pharmaceutically acceptable salt or solvate of any thereof.
   31. The pharmaceutical composition according to Clause 30, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).
   32. The pharmaceutical composition according to any of Clauses 22 to 31, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.
   33. A kit comprising:
   a tropane CCR5/CCL5 interaction inhibitor; and
   a statin.
      34. The kit according to Clause 33, wherein the tropane CCR5/CCL5 interaction inhibitor is described by the formula:

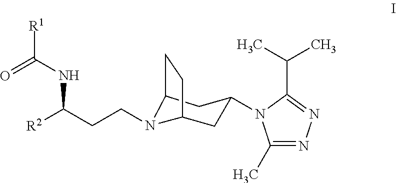

or a pharmaceutically acceptable salt or solvate thereof,
   wherein:
R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms.
   35. The kit according to Clause 34, wherein the tropane CCR5/CCL5 interaction inhibitor is described by the formula:

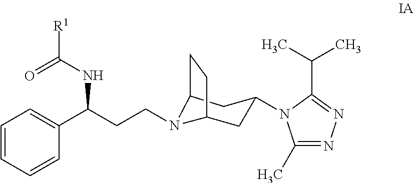

or a pharmaceutically acceptable salt or solvate thereof, wherein:
   R¹ is either C₃₋₆ cycloalkyl optionally substituted by one or more fluorine atoms, or C₁₋₆ alkyl optionally substituted by one or more fluorine atoms.
      36. The kit according to Clause 35, wherein R¹ is either C₄₋₆ cycloalkyl optionally substituted by one or two fluorine atoms, or C₁₋₄ alkyl optionally substituted by from one to three fluorine atoms.
      37 The kit according to Clause 36, wherein R¹ is either cyclobutyl, cyclopentyl, 4,4-difluorocyclohexyl or 3,3,3-trifluoropropyl.
      38. The kit according to Clause 34, wherein R² is phenyl optionally substituted by 1 or 2 fluorine atoms.
      39. The kit according to Clause 38, wherein R² is phenyl or monofluorophenyl.
      40. The kit according to Clause 39, wherein R² is phenyl or 3-fluorophenyl.
      41. The kit according to Clause 33, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl}cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide
or a pharmaceutically acceptable salt or solvate of any thereof.
   42. The kit according to Clause 41, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).
   43. The kit according to any of Clauses 33 to 42, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating a subject for long-haul COVID, the method comprising:
administering to the subject a tropane CCR5/CCL5 interaction inhibitor in combination with a statin to treat the subject for long-haul COVID.

2. The method according to claim 1, wherein the tropane CCR5/CCL5 interaction inhibitor is described by the formula: 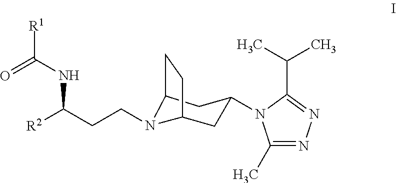
or a pharmaceutically acceptable salt or solvate thereof,
wherein:
R¹ is C₃₋₆ cycloalkyl optionally substituted by one or more fluorine atoms, or C₁₋₆ alkyl optionally substituted by one or more fluorine atoms, or C₃₋₆ cycloalkylmethyl optionally ring-substituted by one or more fluorine atoms; and
R² is phenyl optionally substituted by one or more fluorine atoms.

3. The method according to claim 2, wherein the tropane CCR5/CCL5 interaction inhibitor is described by the formula: 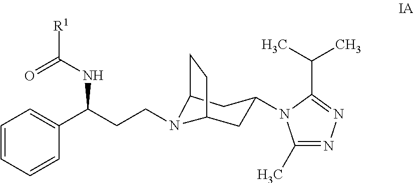
or a pharmaceutically acceptable salt or solvate thereof, wherein:
R¹ is either C₃₋₆ cycloalkyl optionally substituted by one or more fluorine atoms, or C₁₋₆ alkyl optionally substituted by one or more fluorine atoms.

4. The method according to claim 3, wherein R¹ is either C₄₋₆ cycloalkyl optionally substituted by one or two fluorine atoms, or C₁₋₄ alkyl optionally substituted by from one to three fluorine atoms.

5. The method according to claim 4, wherein R¹ is either cyclobutyl, cyclopentyl, 4,4-difluorocyclohexyl or 3,3,3-trifluoropropyl.

6. The method according to claim 2, wherein R² is phenyl optionally substituted by 1 or 2 fluorine atoms.

7. The method according to claim 6, wherein R² is phenyl or monofluorophenyl.

8. The method according to claim 7, wherein R² is phenyl or 3-fluorophenyl.

9. The method according to claim 1, wherein the tropane CCR5/CCL5 interaction inhibitor is selected from the group consisting of:
N-{(1S)-3-[3-(3-Isopropyl-5-methyl-4H-1,2,4-triazol-4-yl)-exo-8-azabicyclo[3.2.1]oct-8-yl]-1-phenylpropyl}cyclobutanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}cyclopentanecarboxamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4,4-trifluorobutanamide;
N-{(1S)-3-[3-(3-Isopropyl-5-methyl-4H-1,2,4-triazol-4-yl)-exo-8-azabicyclo[3.2.1]oct-8-yl]-1-phenylpropyl}-4,4-difluorocyclohexanecarboxamide;
N-{(1S)-3-[3-(3-Isopropyl-5-methyl-4H-1,2,4-triazol-4-yl)-exo-8-azabicyclo[3.2.1]oct-8-yl]-1-(3-fluorophenyl)propyl}-4,4-difluorocyclohexanecarboxamide; and
4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide
or a pharmaceutically acceptable salt or solvate of any thereof.

10. The method according to claim 9, wherein the tropane CCR5/CCL5 interaction inhibitor is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide (maraviroc).

11. The method according to any of the preceding claims, wherein the statin is selected from the group consisting of atorvastatin, cerivastatin, fluvastatin, lovastatin, mevastatin, pitavastatin, pravastatin, rosuvastatin, simvastatin, and pharmaceutically acceptable salts, solvates, stereoisomers, or prodrug derivatives thereof.

12. The method according to any of the preceding claims, wherein the tropane CCR5/CCL5 interaction inhibitor and statin are administered sequentially to the subject.

13. The method according to any of claims 1 to 11, wherein the tropane CCR5/CCL5 interaction inhibitor and statin are administered simultaneously to the subject.

14. A pharmaceutical composition comprising:
a tropane CCR5/CCL5 interaction inhibitor; and
a statin.

15. A kit comprising:
a tropane CCR5/CCL5 interaction inhibitor; and
a statin.
